SEQUENCE LISTING (121 sequences)

SEQ ID NO: 1 - protein, 5 aa, unknown
  Calcium binding motif
DXNDN

SEQ ID NO: 2 - protein, 4 aa, unknown
  Calcium binding motif
LDRE

SEQ ID NO: 3 - protein, 9 aa, Artificial Sequence
  VE-cadherin modulating agent
XFXXDAEXG

SEQ ID NO: 4 - protein, 4 aa, unknown
  VE-cadherin sequence used in a modulating agent
VDAE

SEQ ID NO: 5 - protein, 4 aa, unknown
  VE-cadherin sequence used in a modulating agent
DAET

SEQ ID NO: 6 - protein, 5 aa, unknown
  VE-cadherin sequence used in a modulating agent
RVDAE

SEQ ID NO: 7 - protein, 5 aa, unknown
  VE-cadherin sequence used in a modulating agent
VDAET

SEQ ID NO: 8 - protein, 6 aa, unknown
  VE-cadherin sequence used in a modulating agent
RVDAET

SEQ ID NO: 9 - protein, 5 aa, unknown
  VE-cadherin sequence used in a modulating agent
DAETG

SEQ ID NO: 10 - protein, 6 aa, unknown
  VE-cadherin sequence used in a modulating agent
VDAETG

SEQ ID NO: 11 - protein, 7 aa, unknown
  VE-cadherin sequence used in a modulating agent
RVDAETG

SEQ ID NO: 12 - protein, 6 aa, unknown
  VE-cadherin sequence used in a modulating agent
FRVDAE

SEQ ID NO: 13 - protein, 7 aa, unknown
  VE-cadherin sequence used in a modulating agent
FRVDAET

SEQ ID NO: 14 - protein, 8 aa, unknown
  VE-cadherin sequence used in a modulating agent
FRVDAETG

SEQ ID NO: 15 - protein, 7 aa, unknown
  VE-cadherin sequence used in a modulating agent
VFRVDAE

SEQ ID NO: 16 - protein, 8 aa, unknown
  VE-cadherin sequence used in a modulating agent
VFRVDAET

SEQ ID NO: 17 - protein, 9 aa, unknown
  VE-cadherin sequence used in a modulating agent
VFRVDAETG

SEQ ID NO: 18 - protein, 15 aa, unknown
  VE-cadherin sequence used in a modulating agent
FRVDAETGDVFAIER

SEQ ID NO: 19 - protein, 10 aa, UNKNOWN
  Representative  linear peptide modulating agent
VFRVDAETGD

SEQ ID NO: 20 - protein, 4 aa, UNKNOWN
  Calcium binding motif
XDXE

SEQ ID NO: 21 - protein, 5 aa, UNKNOWN
  Calcium binding motif
DXXDX

SEQ ID NO: 22 - protein, 4 aa, UNKNOWN
  Calcium binding motif
MDRE

SEQ ID NO: 23 - protein, 4 aa, UNKNOWN
  Calcium binding motif
LDFE

SEQ ID NO: 24 - protein, 4 aa, UNKNOWN
  Calcium binding motif
LDYE

SEQ ID NO: 25 - protein, 4 aa, UNKNOWN
  Calcium binding motif
IDRE

SEQ ID NO: 26 - protein, 4 aa, UNKNOWN
  Calcium binding motif
VDRE

SEQ ID NO: 27 - protein, 4 aa, UNKNOWN
  Calcium binding motif
IDFE

SEQ ID NO: 28 - protein, 11 aa, UNKNOWN
  Thrid calcium binding motif with most cadherin repeats
XXXXDXNDXXP

SEQ ID NO: 29 - protein, 8 aa, Artificial Sequence
  CAR sequence derived from EC1 of human VE-cadherin
VFRDAETG

SEQ ID NO: 30 - protein, 5 aa, Artificial Sequence
  Preferred cyclic peptide that may be used as a modulating agent without modification or may be incorporated with a modulating agent.
CDAEC

SEQ ID NO: 31 - protein, 6 aa, Artificial Sequence
  Preferred cyclic peptide that may be used as a modulating agent without modification or may be incorporated with a modulating agent.
CVDAEC

SEQ ID NO: 32 - protein, 6 aa, Artificial Sequence
  Preferred cyclic peptide that may be used as a modulating agent without modification or may be incorporated with a modulating agent.
CDAETC

SEQ ID NO: 33 - protein, 7 aa, Artificial Sequence
  Preferred cyclic peptide that may be used as a modulating agent without modification or may be incorporated with a modulating agent.
CRVDAEC

SEQ ID NO: 34 - protein, 7 aa, Artificial Sequence
  Preferred cyclic peptide that may be used as a modulating agent without modification or may be incorporated with a modulating agent.
CVDAETC

SEQ ID NO: 35 - protein, 8 aa, Artificial Sequence
  Preferred cyclic peptide that may be used as a modulating agent without modification or may be incorporated with a modulating agent.
CRVDAETC

SEQ ID NO: 36 - protein, 7 aa, Artificial Sequence
  Preferred cyclic peptide that may be used as a modulating agent without modification or may be incorporated with a modulating agent.
CDAETGC

SEQ ID NO: 37 - protein, 8 aa, Artificial Sequence
  Preferred cyclic peptide that may be used as a modulating agent without modification or may be incorporated with a modulating agent.
CCDAETGC

SEQ ID NO: 38 - protein, 9 aa, Artificial Sequence
  Preferred cyclic peptide that may be used as a modulating agent without modification or may be incorporated with a modulating agent.
CRVDAETGC

SEQ ID NO: 39 - protein, 8 aa, Artificial Sequence
  Preferred cyclic peptide that may be used as a modulating agent without modification or may be incorporated with a modulating agent.
CFRVDAEC

SEQ ID NO: 40 - protein, 9 aa, Artificial Sequence
  Preferred cyclic peptide that may be used as a modulating agent without modification or may be incorporated with a modulating agent.
CFRVDAETC

SEQ ID NO: 41 - protein, 10 aa, Artificial Sequence
  Preferred cyclic peptide that may be used as a modulating agent without modification or may be incorporated with a modulating agent.
CFRVDAETGC

SEQ ID NO: 42 - protein, 9 aa, Artificial Sequence
  Preferred cyclic peptide that may be used as a modulating agent without modification or may be incorporated with a modulating agent.
CVFRVDAEC

SEQ ID NO: 43 - protein, 10 aa, Artificial Sequence
  Preferred cyclic peptide that may be used as a modulating agent without modification or may be incorporated with a modulating agent.
CVFRVDAETC

SEQ ID NO: 44 - protein, 11 aa, Artificial Sequence
  Preferred cyclic peptide that may be used as a modulating agent without modification or may be incorporated with a modulating agent.
CVFRVDAETGC

SEQ ID NO: 45 - protein, 5 aa, Artificial Sequence
  Preferred cyclic peptide that may be used as a modulating agent without modification or may be incorporated with a modulating agent.
DDAEK

SEQ ID NO: 46 - protein, 6 aa, Artificial Sequence
  Preferred cyclic peptide that may be used as a modulating agent without modification or may be incorporated with a modulating agent.
DVDAEK

SEQ ID NO: 47 - protein, 7 aa, Artificial Sequence
  Preferred cyclic peptide that may be used as a modulating agent without modification or may be incorporated with a modulating agent.
DRVDAEK

SEQ ID NO: 48 - protein, 8 aa, Artificial Sequence
  Preferred cyclic peptide that may be used as a modulating agent without modification or may be incorporated with a modulating agent.
DFRVDAEK

SEQ ID NO: 49 - protein, 9 aa, Artificial Sequence
  Preferred cyclic peptide that may be used as a modulating agent without modification or may be incorporated with a modulating agent.
DVFRVDAEK

SEQ ID NO: 50 - protein, 5 aa, Artificial Sequence
  Preferred cyclic peptide that may be used as a modulating agent without modification or may be incorporated with a modulating agent.
EDAEK

SEQ ID NO: 51 - protein, 6 aa, Artificial Sequence
  Preferred cyclic peptide that may be used as a modulating agent without modification or may be incorporated with a modulating agent.
EVDAEK

SEQ ID NO: 52 - protein, 7 aa, Artificial Sequence
  Preferred cyclic peptide that may be used as a modulating agent without modification or may be incorporated with a modulating agent.
ERVDAEK

SEQ ID NO: 53 - protein, 8 aa, Artificial Sequence
  Preferred cyclic peptide that may be used as a modulating agent without modification or may be incorporated with a modulating agent.
EFRVDAEK

SEQ ID NO: 54 - protein, 9 aa, Artificial Sequence
  Preferred cyclic peptide that may be used as a modulating agent without modification or may be incorporated with a modulating agent.
EVFRVDAEK

SEQ ID NO: 55 - protein, 5 aa, Artificial Sequence
  Preferred cyclic peptide that may be used as a modulating agent without modification or may be incorporated with a modulating agent.
KDAED

SEQ ID NO: 56 - protein, 6 aa, Artificial Sequence
  Preferred cyclic peptide that may be used as a modulating agent without modification or may be incorporated with a modulating agent.
KVDAED

SEQ ID NO: 57 - protein, 6 aa, Artificial Sequence
  Preferred cyclic peptide that may be used as a modulating agent without modification or may be incorporated with a modulating agent.
KDAETD

SEQ ID NO: 58 - protein, 7 aa, Artificial Sequence
  Preferred cyclic peptide that may be used as a modulating agent without modification or may be incorporated with a modulating agent.
KRVDAED

SEQ ID NO: 59 - protein, 7 aa, Artificial Sequence
  Preferred cyclic peptide that may be used as a modulating agent without modification or may be incorporated with a modulating agent.
KVDAETD

SEQ ID NO: 60 - protein, 8 aa, Artificial Sequence
  Preferred cyclic peptide that may be used as a modulating agent without modification or may be incorporated with a modulating agent.
KRVDAETD

SEQ ID NO: 61 - protein, 7 aa, Artificial Sequence
  Preferred cyclic peptide that may be used as a modulating agent without modification or may be incorporated with a modulating agent.
KDAETGD

SEQ ID NO: 62 - protein, 8 aa, Artificial Sequence
  Preferred cyclic peptide that may be used as a modulating agent without modification or may be incorporated with a modulating agent.
KVDAETGD

SEQ ID NO: 63 - protein, 9 aa, Artificial Sequence
  Preferred cyclic peptide that may be used as a modulating agent without modification or may be incorporated with a modulating agent.
KRVDAETGD

SEQ ID NO: 64 - protein, 8 aa, Artificial Sequence
  Preferred cyclic peptide that may be used as a modulating agent without modification or may be incorporated with a modulating agent.
KFRVDAED

SEQ ID NO: 65 - protein, 9 aa, Artificial Sequence
  Preferred cyclic peptide that may be used as a modulating agent without modification or may be incorporated with a modulating agent.
KFRVDAETD

SEQ ID NO: 66 - protein, 10 aa, Artificial Sequence
  Preferred cyclic peptide that may be used as a modulating agent without modification or may be incorporated with a modulating agent.
KFRVDAETGD

SEQ ID NO: 67 - protein, 9 aa, Artificial Sequence
  Preferred cyclic peptide that may be used as a modulating agent without modification or may be incorporated with a modulating agent.
KVFRVDAED

SEQ ID NO: 68 - protein, 10 aa, Artificial Sequence
  Preferred cyclic peptide that may be used as a modulating agent without modification or may be incorporated with a modulating agent.
KVFRVDAETD

SEQ ID NO: 69 - protein, 11 aa, Artificial Sequence
  Preferred cyclic peptide that may be used as a modulating agent without modification or may be incorporated with a modulating agent.
KVFRVDAETGD

SEQ ID NO: 70 - protein, 5 aa, Artificial Sequence
  Preferred cyclic peptide that may be used as a modulating agent without modification or may be incorporated with a modulating agent.
VDAEK

SEQ ID NO: 71 - protein, 5 aa, Artificial Sequence
  Preferred cyclic peptide that may be used as a modulating agent without modification or may be incorporated with a modulating agent.
IDAES

SEQ ID NO: 72 - protein, 5 aa, Artificial Sequence
  Preferred cyclic peptide that may be used as a modulating agent without modification or may be incorporated with a modulating agent.
VDAES

SEQ ID NO: 73 - protein, 5 aa, Artificial Sequence
  Preferred cyclic peptide that may be used as a modulating agent without modification or may be incorporated with a modulating agent.
DAETG

SEQ ID NO: 74 - protein, 6 aa, Artificial Sequence
  Preferred cyclic peptide that may be used as a modulating agent without modification or may be incorporated with a modulating agent.
VDAETG

SEQ ID NO: 75 - protein, 5 aa, Artificial Sequence
  Preferred cyclic peptide that may be used as a modulating agent without modification or may be incorporated with a modulating agent.
KDAEE

SEQ ID NO: 76 - protein, 5 aa, Artificial Sequence
  Preferred cyclic peptide that may be used as a modulating agent without modification or may be incorporated with a modulating agent.
KVDAE

SEQ ID NO: 77 - protein, 6 aa, Artificial Sequence
  Preferred cyclic peptide that may be used as a modulating agent without modification or may be incorporated with a modulating agent.
KDAETE

SEQ ID NO: 78 - protein, 6 aa, Artificial Sequence
  Preferred cyclic peptide that may be used as a modulating agent without modification or may be incorporated with a modulating agent.
KRVDAE

SEQ ID NO: 79 - protein, 7 aa, Artificial Sequence
  Preferred cyclic peptide that may be used as a modulating agent without modification or may be incorporated with a modulating agent.
KVDAETE

SEQ ID NO: 80 - protein, 8 aa, Artificial Sequence
  Preferred cyclic peptide that may be used as a modulating agent without modification or may be incorporated with a modulating agent.
KRVDAETE

SEQ ID NO: 81 - protein, 7 aa, Artificial Sequence
  Preferred cyclic peptide that may be used as a modulating agent without modification or may be incorporated with a modulating agent.
KDAETGE

SEQ ID NO: 82 - protein, 8 aa, Artificial Sequence
  Preferred cyclic peptide that may be used as a modulating agent without modification or may be incorporated with a modulating agent.
KVDAETGE

SEQ ID NO: 83 - protein, 9 aa, Artificial Sequence
  Preferred cyclic peptide that may be used as a modulating agent without modification or may be incorporated with a modulating agent.
KRVDAETGE

SEQ ID NO: 84 - protein, 7 aa, Artificial Sequence
  Preferred cyclic peptide that may be used as a modulating agent without modification or may be incorporated with a modulating agent.
KFRVDAE

SEQ ID NO: 85 - protein, 9 aa, Artificial Sequence
  Preferred cyclic peptide that may be used as a modulating agent without modification or may be incorporated with a modulating agent.
KFRVDAETE

SEQ ID NO: 86 - protein, 10 aa, Artificial Sequence
  Preferred cyclic peptide that may be used as a modulating agent without modification or may be incorporated with a modulating agent.
KFRVDAETGE

SEQ ID NO: 87 - protein, 8 aa, Artificial Sequence
  Preferred cyclic peptide that may be used as a modulating agent without modification or may be incorporated with a modulating agent.
KVFRVDAE

SEQ ID NO: 88 - protein, 10 aa, Artificial Sequence
  Preferred cyclic peptide that may be used as a modulating agent without modification or may be incorporated with a modulating agent.
KVFRVDAETE

SEQ ID NO: 89 - protein, 11 aa, Artificial Sequence
  Preferred cyclic peptide that may be used as a modulating agent without modification or may be incorporated with a modulating agent.
KVFRVDAETGE

SEQ ID NO: 90 - protein, 5 aa, Artificial Sequence
  Preferred cyclic peptide that may be used as a modulating agent without modification or may be incorporated with a modulating agent.
VDAET

SEQ ID NO: 91 - protein, 6 aa, Artificial Sequence
  Preferred cyclic peptide that may be used as a modulating agent without modification or may be incorporated with a modulating agent.
VDAETG

SEQ ID NO: 92 - protein, 5 aa, Artificial Sequence
  Preferred cyclic peptide that may be used as a modulating agent without modification or may be incorporated with a modulating agent.
DAETG

SEQ ID NO: 93 - protein, 5 aa, Artificial Sequence
  Preferred cyclic peptide that may be used as a modulating agent without modification or may be incorporated with a modulating agent.
RVDAE

SEQ ID NO: 94 - protein, 6 aa, Artificial Sequence
  Preferred cyclic peptide that may be used as a modulating agent without modification or may be incorporated with a modulating agent.
RVDAET

SEQ ID NO: 95 - protein, 7 aa, Artificial Sequence
  Preferred cyclic peptide that may be used as a modulating agent without modification or may be incorporated with a modulating agent.
RVDAETG

SEQ ID NO: 96 - protein, 6 aa, Artificial Sequence
  Preferred cyclic peptide that may be used as a modulating agent without modification or may be incorporated with a modulating agent.
FRVDAE

SEQ ID NO: 97 - protein, 7 aa, Artificial Sequence
  Preferred cyclic peptide that may be used as a modulating agent without modification or may be incorporated with a modulating agent.
FRVDAET

SEQ ID NO: 98 - protein, 8 aa, Artificial Sequence
  Preferred cyclic peptide that may be used as a modulating agent without modification or may be incorporated with a modulating agent.
FRVDAETG

SEQ ID NO: 99 - protein, 7 aa, Artificial Sequence
  Preferred cyclic peptide that may be used as a modulating agent without modification or may be incorporated with a modulating agent.
VFRVDAE

SEQ ID NO: 100 - protein, 8 aa, Artificial Sequence
  Preferred cyclic peptide that may be used as a modulating agent without modification or may be incorporated with a modulating agent.
VFRVDAET

SEQ ID NO: 101 - protein, 9 aa, Artificial Sequence
  Preferred cyclic peptide that may be used as a modulating agent without modification or may be incorporated with a modulating agent.
VFRVDAETG

SEQ ID NO: 102 - protein, 5 aa, unknown
  Preferred CAR sequence for inclusion with a modulating agent
YIGSR

SEQ ID NO: 103 - protein, 10 aa, unknown
  Preferred CAR sequence for inclusion with a modulating agent
KYSFNYDGSE

SEQ ID NO: 104 - protein, 17 aa, unknown
  Preferred CAR sequence for inclusion with a modulating agent
IWKHKGRDVILKKDVRF

SEQ ID NO: 105 - protein, 4 aa, unknown
  Preferred CAR sequence for inclusion with a modulating agent
LYHY

SEQ ID NO: 106 - protein, 8 aa, unknown
  Preferred CAR sequence for inclusion with a modulating agent
WXXXXXXG

SEQ ID NO: 107 - protein, 9 aa, unknown
  Preferred CAR sequence for inclusion with a modulating agent
XFXXXXXXG

SEQ ID NO: 108 - protein, 4 aa, unknown
  Representative claudin CAR sequence
IYSY

SEQ ID NO: 109 - protein, 4 aa, unknown
  Representative claudin CAR sequence
TSSY

SEQ ID NO: 110 - protein, 4 aa, unknown
  Representative claudin CAR sequence
VTAF

SEQ ID NO: 111 - protein, 4 aa, unknown
  Representative claudin CAR sequence
VSAF

SEQ ID NO: 112 - protein, 4 aa, unknown
  sequence used for cyclization.
WGGW

SEQ ID NO: 113 - protein, 4 aa, unknown
  E- and/or N-cadherin CAR sequenced used in a modulating agent.
HAVD

SEQ ID NO: 114 - protein, 6 aa, UNKNOWN
  E- and/or N-cadherin CAR sequenced used in a modulating agent.
SHAVSS

SEQ ID NO: 115 - protein, 6 aa, UNKNOWN
  E- and/or N-cadherin CAR sequenced used in a modulating agent.
AHAVDI

SEQ ID NO: 116 - protein, 15 aa, UNKNOWN
  N-cadherin CAR sequence in which antibodies or Fab fragments are directed.
FHLRAHAVDINGNQV

SEQ ID NO: 117 - protein, 48 aa, unknown
  Occuldin CAR sequence in which antibodies or Fab fragments are directed.
GVNPTAQSSGSLYGSQIYALCNQFYTPAATGLYVDQYLYHYCVVDPQE

SEQ ID NO: 118 - protein, 104 aa, Mus musculus
DWIWNQMHIDEEKNESLPHYVGKIKSNVNRQNAKYVLQGEFAGKIFGVDANTGNVLAYERLDREKVSEYFLTALIVDKNTNKNLEQPSSFTVKVHDINDNWPVF

SEQ ID NO: 119 - protein, 104 aa, Homo sapiens
DWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVF

SEQ ID NO: 120 - protein, 104 aa, Bos taurus
DWIWNQMHIDEERNDSLPHYVGKIKSSVDPKKTEYQLRGESAGKVFRVDKNTGDVYALERLDREKISEYHLTALVVDKDSKKNLESPSSFTIKVHDVNDNWPVF

SEQ ID NO: 121 - protein, 104 aa, Sus scrofa
DWIWNQMHIDEEKNGSLPHYVGKIKSSVNHKNTKYQLKGESAGKVFRVDENTGDVYAFERLDREKIPEYQLVALVVDKNTEKNLESPSSFTIKVHDINDNWPVF

(19) United States
(12) Patent Application Publication
(10) Pub. No.: US 2005/0222037 A1
(43) Pub. Date: Oct. 6, 2005

(54) Compounds and methods for modulating VE-cadherin-mediated function

(75) Inventors: Orest W. Blaschuk, Westmount (CA); Barbara J. Gour, Kemptville (CA); James Matthew Symonds, Durham, NC (US); Stephen Byers, Washington, DC (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Adherex Technologies, Inc., Ottawa (CA)

(21) Appl. No.: 11/004,763

(22) Filed: Dec. 3, 2004

Related U.S. Application Data
(63) Continuation-in-part of application No. 10/759,379, filed on Jan. 16, 2004.
Application No. 10/759,379 is a continuation of application No. 09/305,928, filed on May 5, 1999, now Pat. No. 6,682,901.
Application No. 09/305,928 is a continuation-in-part of application No. 09/234,395, filed on Jan. 20, 1999, now Pat. No. 6,680,175.
Application No. 09/234,395 is a continuation-in-part of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.
(63) Continuation-in-part of application No. 10/759,507, filed on Jan. 16, 2004.
Application No. 10/759,507 is a continuation of application No. 09/234,395, filed on Jan. 20, 1999, now Pat. No. 6,680,175.
Application No. 09/234,395 is a continuation-in-part of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.
(63) Continuation-in-part of application No. 10/654,578, filed on Sep. 3, 2003.
Application No. 10/654,578 is a continuation of application No. 09/535,852, filed on Mar. 27, 2000, now Pat. No. 6,638,911.
Application No. 09/535,852 is a continuation-in-part of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.
(63) Continuation-in-part of application No. 10/395,032, filed on Mar. 21, 2003.
Application No. 10/395,032 is a continuation of application No. 09/839,542, filed on Apr. 20, 2001, now Pat. No. 6,569,996.
Application No. 09/839,542 is a division of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.
(63) Continuation-in-part of application No. 10/141,357, filed on May 7, 2002, now abandoned.
Application No. 10/141,357 is a continuation of application No. 09/305,927, filed on May 5, 1999, now Pat. No. 6,433,149.
Application No. 09/305,927 is a continuation-in-part of application No. 09/264,516, filed on Mar. 8, 1999, now Pat. No. 6,593,297.
Application No. 09/264,516 is a continuation-in-part of application No. 09/234,395, filed on Jan. 20, 1999, now Pat. No. 6,680,175.
Application No. 09/234,395 is a continuation-in-part of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.
(63) Continuation-in-part of application No. 10/006,869, filed on Dec. 3, 2001.
Application No. 10/006,869 is a continuation of application No. 09/187,859, filed on Nov. 6, 1998, now Pat. No. 6,358,920.
Application No. 09/187,859 is a continuation-in-part of application No. 09/073,040, filed on May 5, 1998, now Pat. No. 6,472,367.

(51) Int. Cl. A61K 38/08; A61K 38/06

(52) U.S. Cl. 514/14; 514/15; 514/16; 514/17; 514/18; 514/12; 514/13

(57) ABSTRACT

Compositions and methods for modulating VE-cadherin-mediated functions are provided. The compositions and methods employ VE-cadherin modulating agents which generally comprise one or more of: (a) a peptide sequence that is at least 50% identical to a VE-cadherin cell adhesion recognition sequence; (b) a non-peptide mimetic of a VE-cadherin cell adhesion recognition sequence; (c) a substance, such as an antibody or antigen-binding fragment thereof, that specifically binds a VE-cadherin cell adhesion recognition sequence; and/or (d) a polynucleotide encoding a polypeptide that comprises a VE-cadherin cell adhesion recognition sequence or analogue thereof.

### TECHNICAL FIELD

The present invention relates generally to methods for modulating VE-cadherin-mediated functions, and more particularly to the use of DAE-containing modulating agents or analogues thereof or antibodies against these agents for inhibiting or enhancing functions mediated by VE-cadherin.

### BACKGROUND OF THE INVENTION

Cadherins are a superfamily of calcium-dependent cell adhesion molecules (CAMs) (for review, see Munro et al., In: Cell Adhesion and Invasion in Cancer Metastasis, P. Brodt, ed., pp. 17-34, RG Landes Co., Austin Tex., 1996; Rowlands et al (2000) Rev. Reprod. 5: 53-61; Nollet et al. (2000) J. Mol. Biol. 299: 551-572). All cadherins appear to be membrane glycoproteins that generally promote cell adhesion through homophilic interactions (a cadherin on the surface of one cell binds to an identical cadherin on the surface of another cell), although cadherins also appear to be capable of forming heterotypic complexes with one another under certain circumstances and with lower affinity.

There are many different types of cadherins. The most extensively studied group of cadherins is known as the classical, or type I, cadherins. Classical cadherins have been shown to regulate epithelial, endothelial, neural, stem cell and cancer cell adhesion, with different cadherins expressed on different cell types. All classical cadherins have a similar structure. Classical cadherins are composed of five extracellular domains (EC1-EC5), a single hydrophobic domain (TM) that transverses the plasma membrane (PM), and two cytoplasmic domains (CP1 and CP2). The calcium binding motifs DXNDN (SEQ ID NO:1), DXD and LDRE (SEQ ID NO:2) are interspersed throughout the extracellular domains, and each 110 amino acid region that contains such motifs is considered a cadherin repeat. The first extracellular domain (EC1) contains the cell adhesion recognition (CAR) sequence, HAV (His-Ala-Val), along with flanking sequences on either side of the CAR sequence that play a role in conferring specificity. Synthetic peptides containing the HAV sequence and antibodies directed against such peptides have been shown to inhibit classical cadherin-dependent processes (Munro et al., supra; Blaschuk et al., J. Mol. Biol. 211:679-82, 1990; Blaschuk et al., Develop. Biol. 139:227-29, 1990; Alexander et al., J. Cell. Physiol. 156:610-18, 1993, Makrigiannakis. et al. (1999) Am. J. Pathol. 154: 1391-1406; Wilby et al. (1999) Mol. Cell. Neurosci. 14: 66-84; Schnadelbach et al (2000) Mol. Cell. Neurosci. 15: 288-302; Williams et al. (2000) J. Biol. Chem. 275: 4007-4012; Schnadelbach et al. (2001) Mol. Cell. Neurosci. 17: 1084-1093; Erez et al. Exp. Cell Res. 294: 366-78); see also U.S. Pat. Nos. 6,031,072; 6,169,071; 6,417,325).

Cadherins that contain calcium binding motifs within extracellular domain cadherin repeats, but do not contain an HAV CAR sequence, are considered to be nonclassical cadherins. At least six groups of nonclassical cadherins have been identified as well several other cadherins that are not classified within the six groups. These cadherins are also membrane glycoproteins. Type II, or atypical, cadherins include cadherin-11 (OB-cadherin; see Getsios et al., Developmental Dynamics 211:238-247, 1998; Simonneau et al., Cell Adhesion and Communication 3:115-130, 1995; Okazaki et al., J. Biological Chemistry 269:12092-12098, 1994), cadherin-5 (VE-cadherin; see Navarro et al., J. Cell Biology 140:1475-1484, 1998), cadherin-6 (K-cadherin; see Shimoyama et al., Cancer Research 55:2206-2211, 1995; Shimazui et al., Cancer Research 56:3234-3237, 1996; Inoue et al., Developmental Dynamics 211:338-351, 1998; Getsios et al., Developmental Dynamics 211:238-247, 1998), cadherin-7 (see Nakagawa et al., Development 121:1321-1332, 1995), cadherin-8 (see Suzuki et al., Cell Regulation 2:261-270, 1991), cadherin-12 (Br-cadherin; see Tanihara et al., Cell Adhesion and Communication 2:15-26, 1994), cadherin-14 (see Shibata et al., J. Biological Chemistry 272:5236-5240, 1997), cadherin-15 (M-cadherin; see Shimoyama et al., J. Biological Chemistry 273:10011-10018, 1998), and PB-cadherin (see Sugimoto et al., J. Biological Chemistry 271:11548-11556, 1996). For a general review of atypical cadherins, see Redies and Takeichi, Developmental Biology 180:413-423, 1996 and Suzuki et al., Cell Regulation 2:261-270, 1991, Nollet F. et al, (2000) J. Mol. Biol. 299: 551-572.

Other examples of nonclassical cadherins include LI-cadherin (see Berndorff et al., J. Cell Biology 125:1353-1369, 1994), T-cadherin (see Ranscht, U.S. Pat. No. 5,585,351; Tkachuk et al., FEBS Lett. 421:208-212, 1998; Ranscht et al., Neuron 7:391-402, 1991; Sacristan et al., J. Neuroscience Research 34:664-680, 1993; Vestal and Ranscht, J. Cell Biology 119:451-461, 1992; Fredette and Ranscht, J. Neuroscience 14:7331-7346, 1994; Ranscht and Bronner-Fraser, Development 111:15-22, 1991), protocadherins (e.g., protocadherins 42, 43 and 68; see Sano et al., EMBO J. 12:2249-2256, 1993; GenBank Accession Number AF029343), desmocollins (e.g., desmocollins 1, 2, 3 and 4; see King et al., Genomics 18:185-194, 1993; Parker et al., J. Biol. Chem. 266:10438-10445, 1991; King et al., J. Invest. Dermatol. 105:314-321, 1995; Kawamura et al., J. Biol. Chem. 269:26295-26302, 1994), desmogleins (e.g., desmogleins 1 and 2; see Wheeler et al., Proc. Natl. Acad. Sci. USA 88:4796-4800; Koch et al., Eur. J. Cell. Biol. 55:200-208, 1991), and cadherin-related neuronal receptors (see Kohmura et al., Neuron 20:1137-1151, 1998).

Most studies of nonclassical cadherins have focused on atypical or type II cadherins. The structure of these cadherins is similar to that of the type I cadherins, but they do not contain the CAR sequence, HAV. Furthermore, functions mediated by the atypical cadherins may be diverse.

Vascular endothelial cadherin (VE-cadherin also known as cadherin-5) is an endothelial specific cadherin localized at intracellular junctions of essentially all types of endothelium, including the endothelium of blood vessels and of lymphatic vessels. VE-cadherin has been shown to be localized at certain intercellular junctions-adherens junctions (AJ) in cell-to-cell contacts. A number of observations suggest that VE-cadherin is involved in various aspects of vascular biology related to endothelial cell adhesion, angiogenesis, maintenance of vascular integrity and regulation of vascular permeability. In addition to mediating inter-endothelial homotypic cell-cell adhesion, VE-cadherin interacts with and influences the activity of growth factor receptors on the surface of endothelial cells. For instance, VE-cadherin is required for intracellular signals from vascular endothelial growth factor (VEGF) via vascular endothelial growth factor receptor-2 (VEGF-R2) leading to survival of endothelial cells (Carmeliet et al. Cell. 1999 Jul. 23; 98(2):147-57) and VE-cadherin may influence signals from growth factors that regulate the migration and proliferation of endothelial cells (Zanetti et al. Arterioscler Thromb Vasc Biol. 2002 Apr. 1;22(4):617-22). VEGF family members and their receptors are central signalers in the angiogenic process (Carmeliet and Jain, Nature. 2000 Sep. 14; 407(6801):249-57) Collectively, these and other observations underscore the importance of VE-cadherin as a target for the development of novel agents for treating human diseases such as cancer, psoriasis, age-related macular degeneration, ischaemic heart disease, ischaemic limb disease, warts, ulcers, endometriosis, follicular cysts, adhesions, uterine bleeding, atherosclerosis, keloids, ovarian hyperstimulation, peritoneal sclerosis, athritis, asthma, retinopathy, stroke, lymphoproliferative disorders, lymphoedema, thyroid enlargement, intraocular disorders, pulmonary hypertension, healing of bone fractures and obesity.

Notwithstanding these recent advances, there is a need in the art for identifying agents involved in modulating VE-cadherin-dependent functions and processes, such as cell adhesion, and for the development of further methods employing such agents to modulate processes having relevance to human disease conditions, such as cancer cell adhesion, invasion and/or metastasis and angiogenesis-dependent diseases such as those described above. The present invention fulfills these needs and further provides other related advantages.

### SUMMARY OF THE INVENTION

Briefly stated, this invention provides compositions for modulating VE-cadherin-mediated functions and processes including, for example, cell adhesion, angiogenesis, maintenance of vascular integrity, regulation of vascular permeability, and others, and the use of these compositions for treating conditions in which the modulation of one or more such functions and processes is desired.

Therefore, within certain aspects of the invention, modulating agents capable of modulating (i.e., inhibiting or enhancing) one or more functions mediated by a VE-cadherin are provided. Such modulating agents generally: (a) comprise a peptide sequence that is at least 50% identical to a VE-cadherin CAR sequence; and (b) modulate a function or process mediated by a VE-cadherin, such that the modulating agent: (i) detectably inhibits a function that is mediated by the VE-cadherin; or (ii) detectably enhances adhesion of cells that express the VE-cadherin; and (c) contain no more than 85, and preferably no more than 50, consecutive amino acid residues present within a VE-cadherin, such as a naturally occurring VE-cadherin.

In another aspect of the invention, modulating agents are provided that comprise a VE-cadherin CAR sequence as described herein and contain 3-16 amino acid residues, wherein the VE-cadherin CAR sequence comprises the sequence DAE.

In another aspect of the invention, there are provided VE-cadherin modulating agents having the formula:

| | |
|---|---|
| Aaa-Phe-Baa-Ile/Leu/Val-Asp-Ala-Glu- | (SEQ ID NO: 3) |
| Ser/Thr/Asn-Gly | |

wherein Aaa and Baa are independently selected amino acid residues; Ile/Leu/Val is an amino acid that is selected from the group consisting of isoleucine, leucine and valine, and Ser/Thr/Asn is an amino acid that is selected from the group consisting of serine, threonine or asparagine. For other modulating agents as described above, the VE-cadherin CAR sequence consists of at least three consecutive amino acid residues, and preferably at least five consecutive amino acid residues, of a VE-cadherin, wherein the consecutive amino acids are present within a region of the VE-cadherin having the formula recited above. Other modulating agents may comprise at least nine consecutive amino acid residues of a VE-cadherin, wherein the nine consecutive amino acid residues comprise a region having a formula as recited above. Within certain specific embodiments, a modulating agent as described above is a peptide ranging in size from 3 to 50, preferably from 4 to 16, amino acid residues.

Within certain other embodiments, modulating agents of the invention comprise a VE-cadherin CAR sequence that is present within a cyclic peptide. Such cyclic peptides generally have the formula: 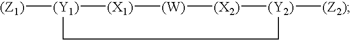
wherein W is the tripeptide DAE, DAN, DKN, DEN, FRV, RVD and/or VDA; wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

Within other aspects, the present invention provides modulating agents comprising polynucleotides encoding a VE-cadherin CAR sequence as described herein, along with expression vectors comprising such polynucleotides and host cells transformed or transfected with such expression vectors.

The present invention further provides modulating agents that comprise an antibody or antigen-binding fragment thereof that specifically binds to a VE-cadherin CAR sequence provided herein and which preferably modulates a VE-cadherin-mediated function.

Within other aspects, the present invention provides modulating agents comprising a non-peptide mimetic of any one of the VE-cadherin CAR sequences provided herein.

Within certain specific embodiments, a modulating agent as provided herein may comprise: (a) one or more VE-cadherin CAR sequences selected from the group consisting of DAE, VDAE (SEQ ID NO: 4), DAET (SEQ ID NO: 5), RVDAE (SEQ ID NO:6), VDAET (SEQ ID NO: 7), RVDAET (SEQ ID NO: 8), DAETG (SEQ ID NO: 9), VDAETG (SEQ ID NO: 10), RVDAETG (SEQ ID NO: 11), FRVDAE (SEQ ID NO: 12), FRVDAET (SEQ ID NO: 13), FRVDAETG (SEQ ID NO: 14), VFRVDAE (SEQ ID NO: 15), VFRVDAET (SEQ ID NO: 16) and VFRVDAETG (SEQ ID NO: 17); or (b) an analogue of any of the foregoing sequences that differs in one or more substitutions, deletions, additions and/or insertions such that that ability of the analogue to modulate a VE-cadherin-mediated function is not substantially diminished. In certain embodiments, the agent may comprise a linear peptide having the sequence N-Ac-VFRVDAETG-NH₂ (SEQ ID NO: 17) or N-Ac-FRVDAETGDVFAIER-NH2 (SEQ ID NO: 18). The VE-cadherin CAR sequence may, but need not, be present within a cyclic peptide.

Any of the modulating agents of the present invention may, within certain embodiments, be linked to one or more of a drug, detectable marker, targeting agent or support material. Alternatively, or in addition, a modulating agent as described above, may further comprise one or more of: (a) a CAR sequence that is specifically recognized by an adhesion molecule other than a VE-cadherin; and/or (b) an antibody or antigen-binding fragment thereof that specifically binds to a CAR sequence that is specifically recognized by an adhesion molecule other than a VE-cadherin. For example, a modulating agent may comprise a CAR sequence from a different non-classical cadherin, such that multiple non-classical cadherin CAR sequences are linked together within the modulating agent.

Within other aspects, the present invention provides pharmaceutical compositions comprising a modulating agent as described above in combination with a physiologically acceptable carrier. Within such compositions, the modulating agent may, but need not, be present within a sustained-release formulation. Such compositions may, within certain embodiments, further comprise a drug and/or a modulator of cell adhesion that comprises one or more of: (a) a CAR sequence that is specifically recognized by an adhesion molecule other than VE-cadherin; and/or (b) an antibody or antigen-binding fragment thereof that specifically binds to a CAR sequence that is specifically recognized by an adhesion molecule other than VE-cadherin.

The present invention further provides, within other aspects, methods for modulating one or more VE-cadherin-mediated functions using the VE-cadherin modulating agents described herein. Such methods generally comprise contacting VE-cadherin-expressing cells with a modulating agent as described herein and thereby modulating a function of VE-cadherin, such as cell adhesion.

Within other aspects, the present invention provides methods for treating, inhibiting or otherwise ameliorating the symptoms of cancer in a mammal, comprising administering to a mammal a modulating agent as described above, wherein the modulating agent inhibits one or more VE-cadherin functions, such as VE-cadherin-mediated cell adhesion. Cancer types which may be treated according to this and other related embodiments include essentially any cancer types which express VE-cadherin and/or which require a blood supply for their growth or survival. Cancers that may express VE-cadherin include, for example, hemangiomas, hemangioendotheliomas, angiosarcomas, Kaposi's sarcoma and epitheloid sarcomas. Cancers which require a blood supply include all tumors that grow beyond the limits of diffusion of nutrients (Folkman, Semin Oncol. (2002) 29(6 Suppl 16):15-8). As VE-cadherin is involved in angiogenesis and maintenance of vascular integrity, cancer types which may be treated with VE-cadherin modulating agents include all those which rely upon a blood supply for their growth or survival, including for example those which are highly vascularized, such as renal adenocarcinomas and glioblastomas. The modulating agent may be administered to the tumor locally, systemically, or by any other suitable means. Certain preferred modulating agents for use within such methods are those that inhibit cell adhesion mediated by VE-cadherin, as described herein.

Within certain preferred aspects, the present invention provides methods for treating metastatic cancer by administering to a mammal one or more modulating agent of the present invention. Essentially any cancer which has metastasized, or has the propensity to metastasize, and which expresses VE-cadherin or requires a blood supply for growth or survival may be treated using the inventive modulating agents described herein, including but not limited to those cancer types recited above. Such agents may be administered to the tumor locally, systemically, or by any other suitable means. Within such methods, the modulating agent may, but need not, be present within a pharmaceutical composition as recited above.

Within certain other aspects, methods are provided for inhibiting adhesion of VE-cadherin-expressing cells in a mammal, comprising administering to a mammal a modulating agent as provided above that inhibits cell adhesion mediated by the VE-cadherin within a cell adhesion assay such as the assays provided herein.

Within further aspects, methods are provided for enhancing the delivery of a drug to a tumor in a mammal, comprising administering to a mammal a modulating agent as described above, wherein the modulating agent inhibits VE-cadherin-mediated cell adhesion. Suitable tumors include, but are not limited to, bladder tumors, ovarian tumors, breast tumors, stomach tumors and kidney tumors and the modulating agent may be administered locally to the tumor or may be administered systemically. Preferred modulating agents for use within such methods are those that inhibit cell adhesion mediated by VE-cadherin, and modulate vascular integrity mediated by VE-cadherin as described herein.

Within other aspects, methods are provided for inhibiting angiogenesis in a mammal, comprising administering to a mammal a modulating agent as described above, wherein the modulating agent inhibits one or more VE-cadherin-mediated functions, such as cell adhesion.

The present invention further provides, within other aspects, methods for inducing apoptosis in a VE-cadherin-expressing cell, comprising contacting a VE-cadherin-expressing cell with a modulating agent as described above, wherein the modulating agent inhibits one or more VE-cadherin-mediated functions such as cell adhesion.

In further aspects, methods are provided for preventing or treating obesity in a mammal, comprising administering to a mammal a modulating agent as described above, wherein the modulating agent inhibits one or more VE-cadherin-mediated functions.

Methods are further provided for stimulating blood vessel regression, comprising administering to a mammal a modulating agent as described above, wherein the modulating agent inhibits one or more VE-cadherin-mediated functions.

The present invention further provides, within other aspects, methods for enhancing drug delivery to the central nervous system of a mammal, comprising administering to a mammal a modulating agent as described above, wherein the modulating agent inhibits one or more VE-cadherin-mediated functions such as maintenance of vascular integrity.

Methods are further provided for increasing vasopermeability in a mammal, comprising administering to a mammal a modulating agent as described above, wherein the modulating agent inhibits one or more VE-cadherin-mediated functions.

Within other aspects, the present invention provides methods for enhancing adhesion of VE-cadherin-expressing cells, comprising contacting VE-cadherin-expressing cells with a modulating agent as described above, wherein the modulating agent enhances VE-cadherin-mediated cell adhesion, wherein the step of contacting is performed under conditions and for a time sufficient to detectably enhance adhesion of the cells. Within certain embodiments, modulating agents for use within such methods are linked to a support molecule or a solid support.

Within related aspects, the present invention provides methods for facilitating wound healing and/or reducing scar tissue in a mammal, comprising contacting a wound in a mammal with a modulating agent as described above, wherein the modulating agent enhances cadherin-mediated cell adhesion. Preferably, the modulating agent enhances VE-cadherin-mediated cell adhesion. Within certain embodiments, modulating agents for use within such methods are linked to a support molecules or a solid support.

Methods are also provided, within other aspects, for enhancing adhesion of foreign tissue implanted within a mammal, comprising contacting a site of implantation of foreign tissue in a mammal with a modulating agent as described above, wherein the modulating agent enhances VE-cadherin-mediated cell adhesion. Such foreign tissue may be a skin graft or organ implant. Within certain embodiments, the modulating agent is linked to a support material, support molecules or a solid support.

Within further aspects, methods are provided for modulating the immune system of a mammal, comprising administering to a mammal a modulating agent as described above, wherein the modulating agent inhibits a VE-cadherin-mediated function.

Within other aspects, the present invention provides methods for preventing pregnancy in a mammal, comprising administering to a mammal a modulating agent as described above, wherein the modulating agent inhibits a VE-cadherin-mediated function.

The present invention further provides methods for detecting the presence of VE-cadherin-expressing cells in a sample, comprising: (a) contacting a sample with an antibody or antigen-binding fragment thereof that binds to a nonclassical CAR sequence as described above under conditions and for a time sufficient to allow formation of an antibody-cadherin complex; and (b) detecting the level of antibody-cadherin complex, and therefrom detecting the presence of VE-cadherin expressing cells in a sample. The antibody may be linked to a support material or a detectable marker such as a fluorescent marker. In certain embodiments, the step of detecting is performed using fluorescence activated cell sorting.

Kits for detecting the presence of cadherin-expressing cells in a sample are also provided. Such kits may comprise: (a) an antibody or antigen-binding fragment thereof that specifically binds to a VE-cadherin CAR sequence; and (b) a detection reagent. Within other aspects, the present invention provides methods for identifying a compound capable of modulating a VE-cadherin-mediated function, comprising: (a) contacting an antibody or antigen-binding fragment thereof that specifically binds to a VE-cadherin CAR sequence as described above with a test compound; and (b) detecting the level of antibody or fragment that binds to the test compound, and therefrom identifying a compound capable of modulating cadherin-mediated cell adhesion.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 provides the amino acid sequences of representative mammalian VE-cadherin EC I domains (SEQ ID NOs:118-121).

FIGS. 2A-2F show human umbilical vein endothelial cells in the presence (FIGS. 2E and 2F) and absence (FIGS. 2A and 2B) of 75 μg/mL of a representative linear peptide modulating agent N-Ac-VFRVDAETGD-NH₂ (SEQ ID NO: 19). FIGS. 2C and 2D show the cells in the presence of 75 μg/mL of a similar peptide without the terminal functional groups. Cells were incubated with peptide for 60 minutes, fixed and immunolabeled with monoclonal antibodies directed against VE-cadherin, and were observed at 400× (A, C and E) and 1000× (B, D and F).

FIGS. 3A-3D show collagen tube formation of human umbilical vein endothelial cells in the presence (FIGS. 3A and 3B) and absence (FIGS. 3C and 3D) of the representative VE-cadherin peptide modulating agent ADH479 (N-Ac-FRVDAETGDVFAIER-NH₂ (SEQ ID NO: 18).

FIGS. 4A-4C show migration properties of human umbilical vein endothelial cells in the presence of 0.25 mg/ml (FIG. 4A), 0.5 mg/ml (FIG. 4B) and 1 mg/ml (FIG. 4C) of the illustrative VE-cadherin peptide modulating agent ADH479 (Ac-FRVDAETGDVFAIER-NH₂; SEQ ID NO: 18)

### DETAILED DESCRIPTION OF THE INVENTION

As noted above, the present invention provides methods for modulating VE-cadherin-mediated functions and/or processes, such as cell adhesion. The present invention is based, in part, on the identification of previously unknown VE-cadherin cell adhesion recognition (CAR) sequences present in naturally occurring VE-cadherins. A modulating agent may generally comprise one or more VE-cadherin CAR sequences (or analogues or mimetics thereof), with or without one or more additional CAR sequences, as described below. Peptide CAR sequences may be present within a linear or cyclic peptide. Alternatively, or in addition, a modulating agent may comprise a polynucleotide encoding a peptide comprising one or more VE-cadherin CAR sequences and/or a modulating agent may comprise a substance (such as an antibody or antigen-binding fragment thereof) that specifically binds to a VE-cadherin CAR sequence.

In general, to modulate a VE-cadherin-mediated function, a cell that expresses a VE-cadherin is contacted with a modulating agent either in vivo or in vitro. Within certain aspects, the methods provided herein inhibit a VE-cadherin-mediated function. Such methods include, for example, methods for treating diseases or other conditions characterized by undesirable cell adhesion, particularly diseases or other conditions associated with VE-cadherin expression, or for facilitating drug delivery to a specific tissue or tumor. Certain methods may inhibit cell adhesion (e.g., endothelial cell adhesion), as well as cancer invasion and metastasis. Alternatively, a modulating agent may, such as when linked to a matrix or to another modulating agent via a linker, be used to enhance a VE-cadherin-mediated function, such as cell adhesion. Such conjugates may be used, for example, to facilitate wound healing or the adhesion of implants.

For example, in certain embodiments further described herein, cancer metastasis may be inhibited (i.e., prevented, diminished in severity or delayed) by the administration of agents that inhibit VE-cadherin-mediated cell adhesion. In other embodiments, blood vessel regression may be stimulated, angiogenesis may be modulated or angiolysis may be stimulated. Such modulating agents may be peptides that correspond to a VE-cadherin CAR sequence, or may be binding agents, such as antibodies and fragments thereof, that specifically recognize a VE-cadherin CAR sequence. In general, within the methods provided herein, a modulating agent is administered to a patient in an amount sufficient to inhibit metastasis, stimulate blood vessel regression, modulate angiogenesis or stimulate angiolysis.

Modulating Agents

As noted above, the term “modulating agent,” as used herein, refers to a molecule comprising at least one of the following components:
(a) a linear or cyclic peptide sequence that is at least 50% identical to a VE-cadherin CAR sequence (i.e., a VE-cadherin CAR sequence or an analogue thereof that retains at least 50% sequence identity);
(b) a mimetic (e.g., peptidomimetic or small molecule mimic) of a VE-cadherin CAR sequence;
(c) a substance, such as an antibody or antigen-binding fragment thereof, that specifically binds a VE-cadherin CAR sequence; and/or
(d) a polynucleotide encoding a polypeptide that comprises a VE-cadherin CAR sequence or analogue thereof.

A modulating agent may consist entirely of one or more of the above elements, or may additionally comprise further peptide and/or non-peptide regions. Additional peptide regions may be derived from a nonclassical cadherin (preferably an extracellular domain that comprises a CAR sequence) and/or may be heterologous. Within certain preferred embodiments, a modulating agent contains no more than 85 consecutive amino acid residues, and preferably no more than 50 consecutive amino acid residues, present within a naturally occurring VE-cadherin.

A modulating agent is further capable of modulating a function or process mediated by a VE-cadherin. Such activity may generally be assessed using, for example, representative assays provided herein. Certain modulating agents inhibit an interaction between VE-cadherin molecules and/or between a VE-cadherin and a different adhesion molecule. Alternatively, to enhance adhesion of VE-cadherin-expressing cells, a modulating agent may comprise an antibody or antigen-binding fragment thereof and/or multiple peptides or mimetics linked to a support material. Such modulating agents may function as a biological glue to bind VE-cadherin-expressing cells, and should result in a detectable enhancement of cell adhesion (preferably an enhancement that is at least as great as that observed for immobilized cadherin or antibody directed against the cadherin).

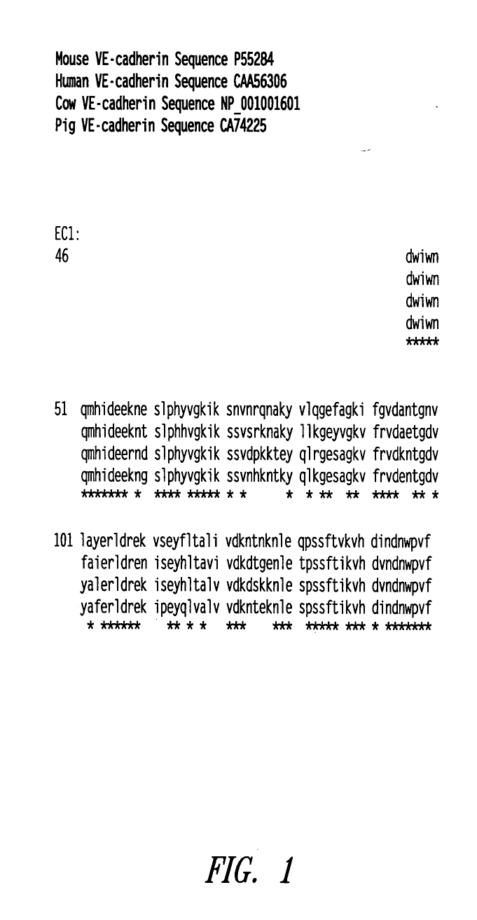

As used herein, the term “VE-cadherin” refers to certain cell adhesion molecules that are expressed by a human or non-human individual, and that are substantially homologous to a known VE-cadherin, such as human VE-cadherin (also known as cadherin-5). Certain representative VE-cadherin EC1 domains are provided in FIG. 1, but the present invention also contemplates the use of VE-cadherin from other organisms, as well as VE-cadherin variants that may have altered amino acid sequences relative to a naturally occurring VE-cadherin molecule, may contain additional amino acids or may be truncated, as described below, provided the variants retain the ability to modulate one or more VE-cadherin functions. VE-cadherin sequences may generally be identified based upon similarity to the sequences provided herein and based upon the presence of VE-cadherin activity, using an assay provided herein.

A VE-cadherin also contains characteristic cadherin repeats, but does not contain the classical cadherin CAR sequence His-Ala-Val (HAV). As used herein, a “cadherin repeat” refers to an amino acid sequence that is approximately 110 amino acid residues in length (generally 100 to 120 residues, preferably 105 to 115 residues), comprises an extracellular domain, and contains three calcium binding motifs (DXD, XDXE and DXXDX; SEQ ID NOs: 20 and 21, respectively) in the same order and in approximately the same position. The presence of an extracellular domain may generally be determined using well known techniques, such as the presence of one or more of: a hydrophilic sequence, a region that is recognized by an antibody, a region that is cleaved by trypsin and/or a potential glycosylation site with the glycosylation motif Asn-X-Ser/Thr. The second calcium binding motif commonly has the sequence LDRE (SEQ ID NO: 2), although variants of this sequence with conservative substitutions are also observed, including MDRE (SEQ ID NO: 22, LDFE (SEQ ID NO:23), LDYE (SEQ ID NO: 24), IDRE (SEQ ID NO: 25), VDRE (SEQ ID NO: 26) and IDFE (SEQ ID NO: 27). Within most cadherin repeats, the third calcium binding motif has the sequence [L,I,V]-X-[L,I,V]-X-D-X-N-D-[N,H]-X-P (SEQ ID NO: 28), wherein residues indicated in brackets may be any one of the recited residues. A preferred third calcium binding motif has the sequence DXNDN (SEQ ID NO: 1), although one or both of the D residues may be replaced by an E. Homology among cadherin repeats is generally at least 20%, preferably at least 30%, as determined by the ALIGN algorithm (Myers and Miller, CABIOS 4:11-17, 1988). Most VE-cadherins comprise at least five cadherin repeats, along with a hydrophobic domain that transverses the plasma membrane and, optionally, one or more cytoplasmic domains.

In certain embodiments, a modulating agent is preferably capable of inhibiting VE-cadherin mediated cell adhesion. Such-activity may generally be assessed using, for example, representative assays provided herein. In general, a modulating agent should inhibit VE-cadherin mediated cell adhesion. Certain modulating agents further inhibit cell adhesion mediated by a different adhesion molecule.

A VE-cadherin CAR sequence, as used herein, is an amino acid sequence that is present in a naturally occurring VE-cadherin and that is capable of detectably modulating a VE-cadherin-mediated function, as described herein. In other words, for example, contacting a VE-cadherin-expressing cell with a peptide comprising a CAR sequence results in a detectable change in VE-cadherin-mediated cell adhesion using at least one of the representative assays provided herein. CAR sequences are generally recognized in vivo by a VE-cadherin or other adhesion molecule (i.e., a molecule that mediates cell adhesion via a receptor on the cell surface), and are necessary for maximal heterophilic and/or homophilic interaction. CAR sequences may be of any length, but generally comprise at least three amino acid residues, preferably 4-16 or 5-9 amino acid residues, and more preferably 3-9 amino acid residues. A peptide modulating agent may comprise any number of amino acid residues, but certain preferred agents comprise about 3-50 residues, while other preferred agents may comprise about 4-16 residues. It will be understood that the number of amino acids present in a modulating agent may vary from these illustrative ranges while still being capable of modulating VE-cadherin function and still being suitable for use in the present invention. For example, the agents may comprise 4-50 residues, 5-50 residues, 6-50 residues, etc., and all values there between.

It has been found, within the context of the present invention, that certain cadherin CAR sequences share the consensus sequence:

| | |
|---|---|
| Aaa-Phe-Baa-Ile/Leu/Val-Asp-Ala-Glu- | (SEQ ID NO: 3) |
| Ser/Thr/Asn-Gly | |

Within the consensus sequence, Aaa and Baa indicate independently selected amino acid residues; “Ile/Leu/Val” indicates an amino acid that is isoleucine, leucine or valine and Ser/Thr/Asn” indicates an amino acid that is serine, threonine or asparagine. CAR sequences specifically provided herein further include portions of such representative CAR sequences, as well as longer polypeptides that comprise at least a portion of such sequences. Additional CAR sequences may be identified based on sequence homology to the CAR sequences provided herein, and based on the ability of a peptide comprising such a sequence to modulate cell adhesion within a representative assay provided herein. Within certain embodiments, a modulating agent comprises at least three consecutive residues, preferably at least five consecutive residues and more preferably at least seven or nine consecutive residues, of a CAR sequence that satisfies the above consensus sequence.

VE-cadherin CAR sequences are generally physically located within the cadherin molecule in or near the binding site of an adhesion molecule (i.e., within 10 amino acids, and preferably within 5 amino acids, of such a binding site). The location of a binding site may generally be determined using well known techniques, such as evaluating the ability of a portion of the VE-cadherin to bind to another VE-cadherin molecule. Any standard binding assay may be employed for such an evaluation. Recognition of a CAR sequence by VE-cadherin results in a measurable effect on cell adhesion. Peptides comprising a CAR sequence generally inhibit such a function.

Certain preferred VE-cadherin CAR sequences comprise 3-9 amino acid residues of the CAR sequence VFRVDAETG (SEQ ID NO: 29), derived from EC1 of human VE-cadherin. For example, a CAR sequence may comprise 3, 4 or 5 residues of this sequence. In general, a VE-cadherin CAR sequence comprises at least the sequence DAE, and in certain more particular embodiments will include at least residues 5-7 of the CAR sequence VFRVDAETG (SEQ ID NO: 29).

Representative VE-cadherin CAR sequences comprise one or more of the peptide sequences DAE, VDAE (SEQ ID NO: 4), DAET (SEQ ID NO: 5), RVDAE (SEQ ID NO:6), VDAET (SEQ ID NO:7), RVDAET (SEQ ID NO:8), DAETG (SEQ ID NO:9), VDAETG (SEQ ID NO:10), RVDAETG (SEQ ID NO:1 1), FRVDAE (SEQ ID NO:12), FRVDAET (SEQ ID NO:13), FRVDAETG (SEQ ID NO:14), VFRVDAE (SEQ ID NO:15), VFRVDAET (SEQ ID NO:16), VFRVDAETG (SEQ ID NO: 17), FRV, RVD, VDA, FRVD (SEQ ID NO: ______), FRVDA (SEQ ID NO: ______), and RVDA (SEQ ID NO: ______). Linear peptides having such sequences may be modified at the N- and/or C-termini.

A modulating agent may contain a greater number of consecutive residues derived from a VE-cadherin. In addition, further flanking sequences may be included to enhance specificity. Such flanking sequences may be identified based on the sequences provided in FIG. 1, for example, or based on published sequences for VE-cadherin molecules. To achieve specificity (i.e., modulation of VE-cadherin-mediated cell adhesion or other function that is enhanced relative to the modulation of a function mediated by a different cadherin), the addition of 2 to 5 flanking residues (preferably at least one residue on either side of the CAR sequence) is generally sufficient. Specificity may be evaluated using assays for the ability to modulate functions mediated by VE-cadherins, as described herein.

As noted above, modulating agents as described herein may comprise an analogue or mimetic of a VE-cadherin CAR sequence. An analogue generally retains at least 50% identity to a native VE-cadherin CAR sequence, and modulates a VE-cadherin-mediated function, such as cell adhesion as described herein. Such analogues preferably contain at least three consecutive residues of, and more preferably at least five consecutive residues of a VE-cadherin CAR sequence. An analogue may contain any of a variety of amino acid substitutions, additions, insertions, deletions and/or modifications (e.g., side chain modifications). Preferred amino acid substitutions are conservative. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and argimne; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; and (5) Phe, Tyr, Trp, His. The critical determining feature of a VE-cadherin CAR sequence analogue is the ability to modulate a VE-cadherin-mediated function, which may be evaluated using the representative assays provided herein.

A mimetic is a non-peptidyl compound that is conformationally similar to a VE-cadherin CAR sequence, such that it modulates a VE-cadherin-mediated function, such as cell adhesion. Such mimetics may be designed based on techniques that evaluate the three dimensional structure of the peptide. For example, Nuclear Magnetic Resonance spectroscopy (NMR) and computational techniques may be used to determine the conformation of a VE-cadherin CAR sequence. NMR is widely used for structural analyses of both peptidyl and non-peptidyl compounds. Nuclear Overhauser Enhancements (NOE's), coupling constants and chemical shifts depend on the conformation of a compound. NOE data provides the interproton distance between protons through space and can be used to calculate the lowest energy conformation for the VE-cadherin CAR sequence. This information can then be used to design mimetics of the preferred conformation. Linear peptides in solution exist in many conformations. By using conformational restriction techniques it is possible to fix the peptide in the active conformation. Conformational restriction can be achieved by i) introduction of an alkyl group such as a methyl which sterically restricts free bond rotation; ii) introduction of unsaturation which fixes the relative positions of the terminal and geminal substituents; and/or iii) cyclization, which fixes the relative positions of the sidechains. Mimetics may be synthesized where one or more of the amide linkages has been replaced by isosteres, substituents or groups which have the same size or volume such as —CH₂NH—, —CSNH—, —CH₂S—, —CH═CH—, —CH₂CH₂—, —CONMe- and others. These backbone amide linkages can also be part of a ring structure (e.g., lactam). Mimetics may be designed where one or more of the side chain functionalities of the VE-cadherin CAR sequence are replaced by groups that do not necessarily have the same size or volume, but have similar chemical and/or physical properties which produce similar biological responses. Other mimetics may be small molecule mimics, which may be readily identified from small molecule libraries, based on the three-dimensional structure of the CAR sequence. It should be understood that, within embodiments described below, an analogue or mimetic may be substituted for a VE-cadherin CAR sequence.

Modulating agents, or peptide portions thereof, may be linear or cyclic peptides. The term “cyclic peptide,” as used herein, refers to a peptide or salt thereof that comprises (1) an intramolecular covalent bond between two non-adjacent residues and (2) at least one VE-cadherin CAR sequence or an analogue thereof. The intramolecular bond may be a backbone to backbone, side-chain to backbone or side-chain to side-chain bond (i.e., terminal functional groups of a linear peptide and/or side chain functional groups of a terminal or interior residue may be linked to achieve cyclization). Preferred intramolecular bonds include, but are not limited to, disulfide, amide and thioether bonds. One or more VE-cadherin CAR sequences, or an analogue or mimetic thereof, may be incorporated into a cyclic peptide, with or without one or more other adhesion molecule binding sites. Additional adhesion molecule binding sites are described in greater detail below.

The size of a cyclic peptide ring generally ranges from 5 to about 15 residues, preferably from 5 to 10 residues. Additional residue(s) may be present on the N-terminal and/or C-terminal side of a VE-cadherin CAR sequence, and may be derived from sequences that flank a VE-cadherin CAR sequence, with or without amino acid substitutions and/or other modifications. Alternatively, additional residues present on one or both sides of the CAR sequence(s) may be unrelated to an endogenous sequence (e.g., residues that facilitate cyclization, purification or other manipulation and/or residues having a targeting or other function).

Within certain embodiments, a modulating agent may comprise a cyclic peptide that contains a VE-cadherin CAR sequence as provided herein (or a portion of such a CAR sequence). Certain illustrative cyclic peptides have the formula:

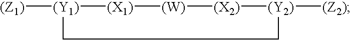

Within this formula, W is the tripeptide DAE, DAN, DKN, DEN, FRV, RVD or VDA; X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; Y₁ and Y₂ are independently selected from the group consisting of amino acid residues, and wherein a covalent bond is formed between residues Y₁ and Y₂; and Z₁ and Z₂ are optional, and if present, are independently selected from the group consisting of amino acid residues and combinations thereof in which the residues are linked by peptide bonds.

Cyclic peptides may comprise any of the above CAR sequence(s). Such cyclic peptides may be used as modulating agents without modification, or may be incorporated into a modulating agent. For example, cyclic peptides may comprise any of the above VE-cadherin CAR sequence(s). Representative cyclic peptides include: CDAEC (SEQ ID NO:30), CVDAEC (SEQ ID NO: 31), CDAETC (SEQ ID NO:32), CRVDAEC (SEQ ID NO:33), CVDAETC (SEQ ID NO:34), CRVDAETC (SEQ ID NO:35), CDAETGC (SEQ ID NO:36), CCDAETGC (SEQ ID NO:37), CRVDAETGC (SEQ ID NO:38), CFRVDAEC (SEQ ID NO:39), CFRVDAETC (SEQ ID NO:40), CFRVDAETGC (SEQ ID NO:41), CVFRVDAEC (SEQ ID NO:42), CVFRVDAETC (SEQ ID NO:43), CVFRVDAETGC (SEQ ID NO:44), DDAEK (SEQ ID NO:45), DVDAEK (SEQ ID NO:46), DRVDAEK (SEQ ID NO:47), DFRVDAEK (SEQ ID NO:48), DVFRVDAEK (SEQ ID NO:49), EDAEK (SEQ ID NO:50), EVDAEK (SEQ ID NO:51), ERVDAEK (SEQ ID NO:52), EFRVDAEK (SEQ ID NO:53), EVFRVDAEK (SEQ ID NO:54), KDAED (SEQ ID NO:55), KVDAED (SEQ ID NO:56), KDAETD (SEQ ID NO:57), KRVDAED(SEQ ID NO:58), KVDAETD (SEQ ID NO:59), KRVDAETD (SEQ ID NO:60), KDAETGD (SEQ ID NO:61), KVDAETGD (SEQ ID NO:62), KRVDAETGD (SEQ ID NO:63), KFRVDAED (SEQ ID NO:64), KFRVDAETD (SEQ ID NO:65), KFRVDAETGD (SEQ ID NO:66), KVFRVDAED (SEQ ID NO:67), KVFRVDAETD (SEQ ID NO:68), KVFRVDAETGD (SEQ ID NO:69), VDAEK (SEQ ID NO:70), IDAES (SEQ ID NO:71), VDAES (SEQ ID NO:72), DAETG (SEQ ID NO:73), VDAETG (SEQ ID NO:74), KDAEE (SEQ ID NO:75), KVDAE (SEQ ID NO:76), KDAETE (SEQ ID NO:77), KRVDAE (SEQ ID NO:78), KVDAETE (SEQ ID NO:79), KRVDAETE (SEQ ID NO:80), KDAETGE (SEQ ID NO:81), KVDAETGE (SEQ ID NO:82), KRVDAETGE (SEQ ID NO:83), KFRVDAE (SEQ ID NO:84), KFRVDAETE (SEQ ID NO:85), KFRVDAETGE (SEQ ID NO:86), KVFRVDAE (SEQ ID NO:87), KVFRVDAETE (SEQ ID NO:88), KVFRVDAETGE (SEQ ID NO:89), VDAET (SEQ ID NO:90), VDAETG (SEQ ID NO:91), DAETG (SEQ ID NO:92), RVDAE (SEQ ID NO:93), RVDAET (SEQ ID NO:94), RVDAETG (SEQ ID NO:95), FRVDAE (SEQ ID NO:96), FRVDAET (SEQ ID NO:97), FRVDAETG (SEQ ID NO:98), VFRVDAE (SEQ ID NO:99), VFRVDAET (SEQ ID NO:100), VFRVDAETG (SEQ ID NO:101), FRV, RVD, DAN, DKN, DEN, VDA, FRVD, FRVDA, RVDA, VDA, CFRVC, CRVDC, CDANC, CDKNC, CDENC, CVDAC, CFRVDC, CFRVDAC, CRVDAC and CVDAC. Within the context of the present invention, underlined sequences are cyclized using any suitable method, as described herein.

As noted above, certain preferred modulating agents comprise a peptide (containing a VE-cadherin CAR sequence or an analogue thereof) in which at least one terminal amino acid residue is modified (e.g., the N-terminal amino group is modified by, for example, acetylation or alkoxybenzylation and/or an amide or ester is formed at the C-terminus). It has been found, within the context of the present invention, that the addition of at least one such group to a linear or cyclic peptide modulating agent may improve the ability of the agent to modulate a VE-cadherin-mediated function. Certain preferred agents contain modifications at the N- and C-terminal residues.

The present invention further contemplates VE-cadherin CAR sequences from other organisms. Such CAR sequences may be identified based upon sequence similarity to the CAR sequences provided herein, and the ability to modulate a VE-cadherin-mediated function such as may be confirmed as described herein.

Within certain embodiments, cyclic peptides that contain small CAR sequences (e.g., three residues without significant flanking sequences) may be preferred. Such peptides may contain an N-acetyl group and a C-amide group. Small cyclic peptides may generally be used to specifically modulate adhesion of endothelial and/or other cell types by topical administration or by systemic administration, with or without linking a targeting agent to the peptide, as discussed below.

A modulating agent may contain one VE-cadherin CAR sequence, or multiple CAR sequences that are adjacent to one another (i.e., without intervening sequences) or in close proximity (i.e., separated by peptide and/or non-peptide linkers to give a distance between the VE-cadherin CAR sequences that ranges from about 0.1 to 400 nm). A linker may be any molecule (including peptide and/or non-peptide sequences) that does not contain a CAR sequence and that can be covalently linked to at least two peptide sequences. Using a linker, CAR sequence-containing peptides and other peptide or protein sequences may be joined end-to-end (i.e., the linker may be covalently attached to the carboxyl or amino group of each peptide sequence), and/or via side chains. One linker that can be used for such purposes is H₂N(CH₂)ₙCO₂H, or derivatives thereof, where n ranges from 1 to 4. Other linkers that may be used will be apparent to those of ordinary skill in the art. Peptide and non-peptide linkers may generally be incorporated into a modulating agent using any appropriate method known in the art.

Within embodiments in which enhancement of cell adhesion mediated by a VE-cadherin is desired, a modulating agent may contain multiple VE-cadherin CAR sequences, or antibodies that specifically bind to such sequences, joined by linkers as described above. For enhancers of cadherin function, the linker distance should generally be 400-10,000 nm. One linker that can be used for such purposes is (H2N(CH2)nCO2H)m, or derivatives thereof, where n ranges from 1 to 10 and m ranges from 1 to 4000. For example, if glycine (H2NCH2CO2H) or a multimer thereof is used as a linker, each glycine unit corresponds to a linking distance of 2.45 angstroms, or 0.245 nm, as determined by calculation of its lowest energy conformation when linked to other amino acids using molecular modeling techniques. Similarly, aminopropanoic acid corresponds to a linking distance of 3.73 angstroms, aminobutanoic acid to 4.96 angstroms, aminopentanoic acid to 6.30 angstroms and amino hexanoic acid to 6.12 angstroms. Enhancement of cell adhesion may also be achieved by attachment of multiple modulating agents to a support material, as discussed further below.

Within related embodiments, modulating agents that enhance cell adhesion preferably contain multiple CAR sequence motifs, provided such sequences are adjacent to one another in spatial orientation relative to one another that is effective for engaging two cadherin molecules, and thereby enhances cadherin-mediated adhesion and other cadherin-dependent processes. For example, dimeric forms of CAR-containing peptides may be useful in certain embodiments in which enhancement of cadherin-mediated processes is desired. Dimeric forms of DAE-containing cyclic peptides are also useful in the embodiments described herein. For example, cyclic peptides comprising the sequence CYDAE-x-DAEYC, wherein X is 1-10 amino acids in length and Y is 0-10 amino acids in length, or cyclic peptides comprising CDAEC-CDAEC (cyclized), may be particularly preferred in certain embodiments. The spacing between DAE-containing motifs present within a DAE-containing multimer may vary while still giving rise to a desired level of agonist activity. A spacing of 1-10 amino acid residues, preferably 4-10 amino acid residues, between DAE-motifs in a DAE-containing multimer, for example, may be desirable in certain embodiments. Moreover, the degree of agonist activity of a given DAE-containing multimer may vary depending upon the concentration of the agent employed relative to the number of cadherin molecules being targeted in a given sample or subject, i.e., the level of saturation of the system being treated. Representative means for evaluating the agonist activity of a DAE-containing multimer are provided elsewhere herein. Enhancement of cell adhesion may also be achieved by attachment of a single DAE motif, multiple DAE motifs and/or multiple modulating agents to a support molecule or material, as discussed herein. Such modulating agents may additionally comprise one or more CAR sequence for one or more different adhesion molecules (including, but not limited to, other CAMs) and/or one or more antibodies or fragments thereof that bind to such sequences, to enhance cell adhesion mediated by multiple adhesion molecules.

Any VE-cadherin modulating agent or composition comprising a VE-cadherin modulating agent of the present invention may further comprise, in addition to one or more VE-cadherin CAR sequence, one or more CAR sequences derived from a different cell adhesion molecule, one or more antibodies or fragments thereof that bind to such sequences, one or more polynucleotides encoding such sequences, and the like. Linkers may, but need not, be used to separate such CAR sequence(s) and/or antibody sequence(s) from the CAR sequence(s) and/or each other. Such modulating agents may generally be used within methods in which it is desirable to simultaneously disrupt a function mediated by multiple adhesion molecules.

As used herein, an “adhesion molecule” is any molecule that mediates cell adhesion via a receptor on the cell's surface. Adhesion molecules include members of the cadherin gene superfamily including classical cadherins (preferably containing an HAV sequence), desmogleins (Dsg) and desmocollins (Dsc); integrins; members of the immunoglobulin supergene family, such as N-CAM; and other transmembrane proteins, such as occludin and claudin, as well as extracellular matrix proteins such as laminin, fibronectin, collagens, vitronectin, entactin and tenascin.

Preferred CAR sequences for inclusion within a modulating agent include (a) Arg-Gly-Asp (RGD), which is bound by integrins (see Cardarelli et al., J. Biol. Chem. 267:23159-64, 1992); (b) Tyr-Ile-Gly-Ser-Arg (YIGSR; SEQ ID NO:102), which is bound by α6β1 integrin; (c) KYSFNYDGSE (SEQ ID NO: 103), which is bound by N-CAM; (d) the N-CAM heparin-sulfate-binding site IWKHKGRDVILKKDVRF (SEQ ID NO:104); (e) the occludin CAR sequence LYHY (SEQ ID NO: 105); (f) claudin CAR sequences comprising at least four consecutive amino acids present within a claudin region that has the formula: Trp-Lys/Arg-Aaa-Baa-Ser/Ala-Tyr/Phe-Caa-Gly (SEQ ID NO: 106), wherein Aaa, Baa and Caa indicate independently selected amino acid residues; Lys/Arg is an amino acid that is lysine or arginine; Ser/Ala is an amino acid that is serine or alanine; and Tyr/Phe is an amino acid that is tyrosine or phenylalanine; and (g) nonclassical cadherin CAR sequences comprising at least three consecutive amino acids present within a nonclassical cadherin region that has the formula: Aaa-Phe-Baa-Ile/Leu/Val-Asp/Asn/Glu-Caa-Daa-Ser/Thr/Asn-Gly (SEQ ID NO:107), wherein Aaa, Baa, Caa and Daa are independently selected amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine. Representative claudin CAR sequences include IYSY (SEQ ID NO:108), TSSY (SEQ ID NO:______), VTAF (SEQ ID NO:110) and VSAF (SEQ ID NO:111). Representative nonclassical cadherin CAR sequences include the OB-cadherin CAR sequences DDK, EEY, EAQ and QAV; the cadherin-6 CAR sequences EEY, NEN, ESE and DSG; the cadherin-7 CAR sequences DEN, EPK and DAN; the cadherin-8 CAR sequences EEF and NDV; the cadherin- 12 CAR sequences DET and DPK; the cadherin- 14 CAR sequences DDT, DPK and DAN; the cadherin- 15 CAR sequences DKF and DEL; the PB-cadherin CAR sequences EEY, DEL, DPK and DAD; the protocadherin CAR sequences DLV, NRD, DPK and DPS; the dsg CAR sequences NQK, NRN and NKD; the dsc CAR sequences EKD and ERD and the cadherin-related neuronal receptor CAR sequences DPV, DAD, DSV, DSN, DSS, DEK and NEK.

Using linkers, such modulating agents may form linear or branched structures. For example, bi-functional modulating agents that comprise a VE-cadherin CAR sequence joined via a linker to separate CAR sequence(s) may be preferred for certain embodiments. As noted above, in certain embodiments, linkers preferably produce a distance between CAR sequences ranging from 0.1 to 10,000 nm, more preferably ranging from 0.1-400 nm. A separation distance between recognition sites may generally be determined according to the desired function of the modulating agent.

The total number of CAR sequences (including the VE-cadherin CAR sequence, with or without other CAR sequences derived from one or more different adhesion molecules) present within a modulating agent may range from 1 to a large number, such as 100, preferably from 1 to 10, and more preferably from 1 to 5. Peptide modulating agents comprising multiple CAR sequences typically contain from 6 (e.g., DAE-HAV) to about 1000 amino acid residues, preferably from 6 to 50 residues. When non-peptide linkers are employed, each CAR sequence of the modulating agent is present within a peptide that generally ranges in size from 3 to 50 residues in length, preferably from 4 to 25 residues, and more preferably from 5 to 15 residues.

As noted above, modulating agents may be polypeptides or salts thereof, containing only amino acid residues linked by peptide bonds, or may contain non-peptide regions, such as linkers. Peptide regions of a modulating agent may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. The 20 L-amino acids commonly found in proteins are identified herein by the conventional three-letter or one-letter abbreviations, and the corresponding D-amino acids are designated by a lower case one letter symbol.

Modulating agents may also contain rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Preferred derivatives include amino acids having a C-terminal amide group. Residues other than common amino acids that may be present within a modulating agent include, but are not limited to, 2-mercaptoaniline, 2-mercaptoproline, ornithine, diaminobutyric acid, α-aminoadipic acid, m-aminomethylbenzoic acid and α,β-diaminopropionic acid.

Peptide modulating agents (and peptide portions of modulating agents) as described herein may be synthesized by methods well known in the art, including chemical synthesis and recombinant DNA methods. For modulating agents up to about 50 residues in length, chemical synthesis may be performed using solution or solid phase peptide synthesis techniques, in which a peptide linkage occurs through the direct condensation of the α-amino group of one amino acid with the α-carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy. The Boc strategy uses a 1% cross-linked polystyrene resin. The standard protecting group for α-amino functions is the tert-butyloxycarbonyl (Boc) group. This group can be removed with dilute solutions of strong acids such as 25% trifluoroacetic acid (TFA). The next Boc-amino acid is typically coupled to the amino acyl resin using dicyclohexylcarbodiimide (DCC). Following completion of the assembly, the peptide-resin is treated with anhydrous HF to cleave the benzyl ester link and liberate the free peptide. Side-chain functional groups are usually blocked during synthesis by benzyl-derived blocking groups, which are also cleaved by HF. The free peptide is then extracted from the resin with a suitable solvent, purified and characterized. Newly synthesized peptides can be purified, for example, by gel filtration, HPLC, partition chromatography and/or ion-exchange chromatography, and may be characterized by, for example, mass spectrometry or amino acid sequence analysis. In the Boc strategy, C-terminal amidated peptides can be obtained using benzhydrylamine or methylbenzhydrylamine resins, which yield peptide amides directly upon cleavage with HF.

In the procedures discussed above, the selectivity of the side-chain blocking groups and of the peptide-resin link depends upon the differences in the rate of acidolytic cleavage. Orthoganol systems have been introduced in which the side-chain blocking groups and the peptide-resin link are completely stable to the reagent used to remove the α-protecting group at each step of the synthesis. The most common of these methods involves the 9-fluorenylmethyloxycarbonyl (Fmoc) approach. Within this method, the side-chain protecting groups and the peptide-resin link are completely stable to the secondary amines used for cleaving the N-α-Fmoc group. The side-chain protection and the peptide-resin link are cleaved by mild acidolysis. The repeated contact with base makes the Merrifield resin unsuitable for Fmoc chemistry, and p-alkoxybenzyl esters linked to the resin are generally used. Deprotection and cleavage are generally accomplished using TFA.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

Acetylation of the N-terminus can be accomplished by reacting the final peptide with acetic anhydride before cleavage from the resin. C-amidation is accomplished using an appropriate resin such as methylbenzhydrylamine resin using the Boc technology.

Following synthesis of a linear peptide, with or without N-acetylation and/or C-amidation, cyclization may be achieved if desired by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Cyclic peptides produced by this method require purification using standard techniques, but this oxidation is applicable at acid pHs. Oxidizing agents also allow concurrent deprotection/oxidation of suitable S-protected linear precursors to avoid premature, nonspecific oxidation of free cysteine.

DMSO, unlike I₂ and K₃Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation. Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic acid (Mpr), β,β-pentamethylene-β-mercaptopropionic acid (Pmp), 2-mercaptobenzene, 2-mercaptoaniline and 2-mercaptoproline.

It will be readily apparent to those of ordinary skill in the art that, within each of these representative formulas, any of the above thiol-containing residues may be employed in place of one or both of the thiol-containing residues recited.

Within another embodiment, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate.

Methods for forming amide bonds are well known in the art and are based on well established principles of chemical reactivity. Within one such method, carboduimide-mediated lactam formation can be accomplished by reaction of the carboxylic acid with DCC, DIC, EDAC or DCCI, resulting in the formation of an O-acylurea that can be reacted immediately with the free amino group to complete the cyclization. The formation of the inactive N-acylurea, resulting from O→N migration, can be circumvented by converting the O-acylurea to an active ester by reaction with an N-hydroxy compound such as 1-hydroxybenzotriazole, 1-hydroxysuccinimide, 1-hydroxynorbornene carboxamide or ethyl 2-hydroximino-2-cyanoacetate. In addition to minimizing O→N migration, these additives also serve as catalysts during cyclization and assist in lowering racemization. Alternatively, cyclization can be performed using the azide method, in which a reactive azide intermediate is generated from an alkyl ester via a hydrazide. Hydrazinolysis of the terminal ester necessitates the use of a t-butyl group for the protection of side chain carboxyl functions in the acylating component. This limitation can be overcome by using diphenylphosphoryl acid (DPPA), which furnishes an azide directly upon reaction with a carboxyl group. The slow reactivity of azides and the formation of isocyanates by their disproportionation restrict the usefulness of this method. The mixed anhydride method of lactam formation is widely used because of the facile removal of reaction by-products. The anhydride is formed upon reaction of the carboxylate anion with an alkyl chloroformate or pivaloyl chloride. The attack of the amino component is then guided to the carbonyl carbon of the acylating component by the electron donating effect of the alkoxy group or by the steric bulk of the pivaloyl chloride t-butyl group, which obstructs attack on the wrong carbonyl group. Mixed anhydrides with phosphoric acid derivatives have also been successfully used. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active ester” useful in the synthesis of amide bonds. The last few years have witnessed the development of benzotriazolyloxytris-(dimethylamino)phosphonium hexafluorophosphonate (BOP) and its congeners as advantageous coupling reagents. Their performance is generally superior to that of the well established carbodiimide amide bond formation reactions.

Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF. Examples of thiol-containing linkages are shown below:

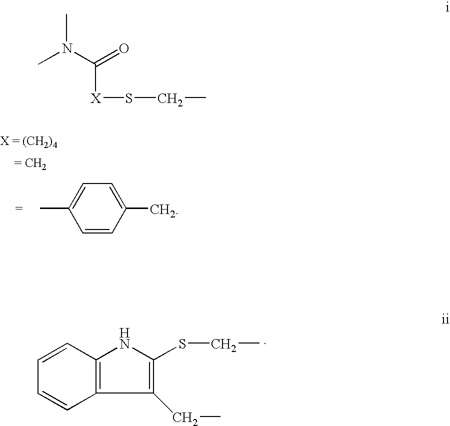

Cyclization may also be achieved using δ₁,δ_{1′}-Ditryptophan (i.e., Ac-Trp-Gly-Gly-Trp-OMe) (SEQ ID NO: 112), as shown below:

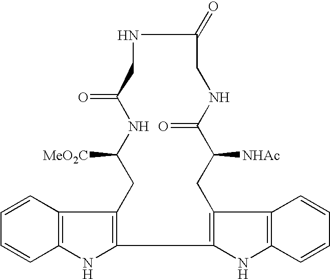

The structures and formulas recited herein are provided solely for the purpose of illustration, and are not intended to limit the scope of the cyclic peptides described herein.

For longer modulating agents, recombinant methods are preferred for synthesis. Within such methods, all or part of a modulating agent can be synthesized in living cells, using any of a variety of expression vectors known to those of ordinary skill in the art to be appropriate for the particular host cell. Suitable host cells may include bacteria, yeast cells, mammalian cells, insect cells, plant cells, algae and other animal cells (e.g., hybridoma, CHO, myeloma). The DNA sequences expressed in this manner may encode portions of a VE-cadherin or other adhesion molecule, or may encode a peptide comprising a VE-cadherin analogue or an antibody fragment that specifically binds to a VE-cadherin CAR sequence. Such DNA sequences may be prepared based on known cDNA or genomic sequences, or from sequences isolated by screening an appropriate library with probes designed based on the sequences of known VE-cadherins. Such screens may generally be performed as described in Sambrook et al., Molecular Cloning: A Laboratory Manual, Cold Spring Harbor Laboratories, Cold Spring Harbor, N.Y., 1989 (and references cited therein). Polymerase chain reaction (PCR) may also be employed, using oligonucleotide primers in methods well known in the art, to isolate nucleic acid molecules encoding all or a portion of an endogenous adhesion molecule. To generate a nucleic acid molecule encoding a desired modulating agent, an endogenous cadherin sequence may be modified using well known techniques. For example, portions encoding one or more CAR sequences may be joined, with or without separation by nucleic acid regions encoding linkers, as discussed above. Alternatively, portions of the desired nucleic acid sequences may be synthesized using well known techniques, and then ligated together to form a sequence encoding the modulating agent.

As noted above, polynucleotides may also function as modulating agents. In general, such polynucleotides should be formulated to permit expression of a polypeptide modulating agent following administration to a mammal. Such formulations are particularly useful for therapeutic purposes, as described below. Those of ordinary skill in the art will appreciate that there are many ways to achieve expression of a polynucleotide within a mammal, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transfected cells) and/or a targeting moiety, such as a gene that encodes a ligand for a receptor on a specific target cell, to render the vector target specific. Targeting may also be accomplished using an antibody, by methods known to those of ordinary skill in the art. Other formulations for polynucleotides for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

As noted above, modulating agent may additionally, or alternatively, comprise a substance such as an antibody or antigen-binding fragment thereof, that specifically binds to a VE-cadherin CAR sequence. As used herein, a substance is said to “specifically bind” to a VE-cadherin CAR sequence (with or without flanking amino acids) if it reacts at a detectable level with a peptide containing that sequence, and does not react detectably with peptides containing a different CAR sequence or a sequence in which the order of amino acid residues in the cadherin CAR sequence and/or flanking sequence is altered. Such antibody binding properties may generally be assessed using an ELISA, which may be readily performed by those of ordinary skill in the art and is described, for example, by Newton et al., Develop. Dynamics 197:1-13, 1993.

Polyclonal and monoclonal antibodies may be raised against a VE-cadherin CAR sequence using conventional techniques. See, e.g., Harlow and Lane, Antibodies: A Laboratory Manual, Cold Spring Harbor Laboratory, 1988. In one such technique, an immunogen comprising the CAR sequence is initially injected into any of a wide variety of mammals (e.g., mice, rats, rabbits, sheep or goats). The smaller immunogens (i.e., less than about 20 amino acids) should be joined to a carrier protein, such as bovine serum albumin or keyhole limpet hemocyanin. Following one or more injections, the animals are bled periodically. Polyclonal antibodies specific for the CAR sequence may then be purified from such antisera by, for example, affinity chromatography using the CAR sequence or antigenic portion thereof coupled to a suitable solid support.

Monoclonal antibodies specific for a VE-cadherin sequence may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity from spleen cells obtained from an animal immunized as described above. The spleen cells are immortalized by, for example, fusion with a myeloma cell fusion partner, preferably one that is syngeneic with the immunized animal. Single colonies are selected and their culture supernatants tested for binding activity against the modulating agent or antigenic portion thereof. Hybridomas having high reactivity and specificity are preferred.

Monoclonal antibodies may be isolated from the supernatants of growing hybridoma colonies, with or without the use of various techniques known in the art to enhance the yield. Contaminants may be removed from the antibodies by conventional techniques, such as chromatography, gel filtration, precipitation and extraction. Antibodies having the desired activity may generally be identified using immunofluorescence analyses of tissue sections, cell or other samples where the target cadherin is localized.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988; see especially page 309) and digested by papain to yield Fab and Fc fragments. The Fab and Fc fragments may be separated by affinity chromatography on protein A bead columns (Harlow and Lane, 1988, pages 628-29).

Evaluation of Modulating Agent Activity

Modulating agents as described above are capable of modulating one or more VE-cadherin-mediated functions, such as cell adhesion, angiogenesis, maintenance of vascular integrity or regulation of vascular permeability. An initial screen for such activity may be performed by evaluating the ability of a modulating agent to bind to VE-cadherin using any binding assay known to those of ordinary skill in the art. For example, a Pharmacia Biosensor machine may be used, as discussed in Jonsson et al., Biotechniques 11:520-27, 1991. A specific example of a technology that measures the interaction of peptides with molecules can be found in Williams et al., J. Biol. Chem. 272, 22349-22354, 1997. Alternatively, real-time BIA (Biomolecular Interaction Analysis) uses the optical phenomenon surface plasmon resonance to monitor biomolecular interactions. The detection depends upon changes in the mass concentration of macromolecules at the biospecific interface, which in turn depends upon the immobilization of test molecule or peptide (referred to as the ligand) to the surface of a Biosensor chip, followed by binding of the interacting molecule (referred to as the analyte) to the ligand. Binding to the chip is measured in real-time in arbitrary units of resonance (RU).

By way of example, surface plasmon resonance experiments may be carried out using a BIAcore X™ Biosensor (Pharmacia Ltd., BIAcore, Uppsala, Sweden). Parallel flow cells of CM 5 sensor chips may be derivatized, using the amine coupling method, with streptavidin (200 μg/ml) in 10 mM Sodium Acetate, pH 4.0, according to the manufacturer's protocol. Approximately 2100-2600 resonance units (RU) of ligand may be immobilized, corresponding to a concentration of about 2.1-2.6 ng/mm². The chips may then coated be with VE-cadherin derivatized to biotin. Any non-specifically bound protein is removed.

To determine binding, test analytes (e.g., peptides containing the VE-cadherin CAR sequence) may be placed in running buffer and passed simultaneously over test and control flow cells. After a period of free buffer flow, any analyte remaining bound to the surface may be removed with, for example, a pulse of 0.1% SDS bringing the signal back to baseline. Specific binding to the derivatized sensor chips may be determined automatically by the system by subtraction of test from control flow cell responses. In general, a modulating agent binds to VE-cadherin at a detectable level within such as assay. The level of binding is preferably at least that observed for the full length VE-cadherin under similar conditions.

The ability to inhibit VE-cadherin-mediated cell function may be evaluated using any of a variety of in vitro assays. It has been found, within the context of the present invention, that VE-cadherin is associated with adhesion of certain cell types, including many endothelial cell types. The ability of an agent to inhibit VE-cadherin mediated function may generally be evaluated in vitro, for example by assaying the effect on adhesion between VE-cadherin-expressing cells (i.e., any type of cell that expresses VE-cadherin at a detectable level, using standard techniques such as immunocytochemical protocols) (e.g., Blaschuk and Farookhi, Dev. Biol. 136:564-567, 1989), such as endothelial cells).

In general, an agent is an inhibitor of cell adhesion if contact of the test cells with the modulating agent results in a discernible disruption of cell adhesion, when such cells are plated under standard conditions that, in the absence of modulating agent, permit cell adhesion. In the presence of modulating agent (e.g., 1 mg/mL), disruption of cell adhesion may be determined visually within 24 hours, by observing retraction of the cells from one another and the substratum.

Alternatively, cells that do not naturally express VE-cadherin may be used within such assays. Such cells may be stably transfected with a polynucleotide (e.g., a cDNA) encoding VE-cadherin, such that VE-cadherin is expressed on the surface of the cell. Expression of the cadherin may be confirmed by assessing adhesion of the transfected cells, in conjunction with immunocytochemical techniques using antibodies directed against the cadherin of interest. The stably transfected cells that aggregate, as judged by light microscopy, following transfection express sufficient levels of VE-cadherin. Preferred cells for use in such assays include L cells, which do not detectably adhere and do not express any cadherin (Nagafuchi et al., Nature 329:341-343, 1987). Following transfection of L cells with a cDNA encoding VE-cadherin, aggregation is observed (see Brier et al., Blood 87:630-641). Modulating agents detectably inhibit such aggregation.

Transfection of cells for use in cell adhesion assays may be performed using standard techniques and published VE-cadherin sequences. For example, a sequence of VE-cadherin may be found within references cited herein and in the GenBank database at accession number X59796 (human cadherin-5).

By way of example, an assay for evaluating a modulating agent for the ability to inhibit VE-cadherin mediated cell adhesion may employ a suitable endothelial cell line. According to a representative procedure, the cells may be isolated from human umbilical veins and cultured on 1% gelatin-coated flask in medium 199 with 20% newborn calf serum (NCS) supplemented with 50 μg/mL endothelial cell growth supplement and 100 μg/mL heparin. Cells may be harvested and replated on glass coverslips and grown to confluency (Corada et al., Blood (2001) 97:1679-1684) and exposed to modulating agent at a concentration of, for example, 1 mg/mL for 24 hours. Cells may be fixed with 95% ethanol for 30 min followed by acetone for 1 min and incubated for 1 hr at 37° C. with antibodies to VE-cadherin e.g. primary antibody for VE-cadherin (Immunotech Marseilles, France) at 1:250 dilution. Coverslips may then be washed with 0.1% milk/PBS solution×3 for 5 min each. Secondary antibody may then be added e.g. goat anti-rabbit-FITC (Zymed, San Francisco, Calif.) at 1:250 dilution for 1 hr at 37° C. Coverslips may then be washed with 0.1 % milk/PBS solution×3 for 5 min each and mounted with anti-quenching solution (e.g. 1 mg/mL phenylenediamine (Sigma, St. Louis, Mo.) in 50% glycerol/50% PBS). Cells may be viewed by fluorescence microscopy. In the absence of modulating agent, human endothelial cells are tightly adherent and VE-cadherin expression is confined to a narrow line along cell-cell contacts. Endothelial cells that are treated with modulating agent may detatch from one another, and disruptions in the integrity of the monolayer may be apparent by the appearance of holes between the cells and by perturbations in the VE-cadherin staining pattern.

Certain modulating agents according to the invention inhibit angiogenesis. The effect of a particular modulating agent on angiogenesis may generally be determined by evaluating the effect of the agent on blood vessel formation. Such a determination may generally be performed, for example, using a chick chorioallantoic membrane assay (Iruela-Arispe et al., Molecular Biology of the Cell 6:327-343, 1995). Briefly, a modulating agent may be embedded in a mesh composed of vitrogen at one or more concentrations (e.g., ranging from about 1 to 100 μg/mesh). The mesh(es) may then be applied to chick chorioallantoic membranes. After 24 hours, the effect of the modulating agent may be determined using computer assisted morphometric analysis. A modulating agent should inhibit angiogenesis by at least 25% at a concentration of 33 μg/mesh.

The effect of a modulating agent on angiogenesis may also be determined by evaluating the effect of the agent on neovascularization in the ex vivo allantois assay (Downs et al. (2001) Developmental Biology 233:347-364; Drake and Fleming (2000) Blood 95:1671-1679). Briefly, a modulating agent may be applied to allantoides that are dissected from 7.5 or 8.5 days postcoitum (dpc) mouse embryos. The effects of the modulating agent on neovascularization in the allantoides may be determined by observating vascular formation. Allantoides that are treated with modulating agents should exhibit signs of inhibited angiogenesis and vascular defects.

Certain modulating agents according to the invention may inhibit endothelial tube formation in vitro. The effect of a particular modulating agent on endothelial tube formation in vitro may generally be determined by evaluating the effect of the agent on tube formation. Such a determination may generally be performed, for example, using a 3D tubulogenesis assay (Corada et al., Blood. 2002, 100:905-911). Briefly, three-dimensional cultures of endothelial cells may be prepared by culturing human umbilical vein endothelial cells in gels of type I collagen in 24-well culture plates. Modulating agents may be applied to the cultures e.g. at 1 mg/mL ranging to 0.1 mg/mL. Capillary tube formation may be followed by phase contrast microscopy. In the absence of modulating agent, endothelial cells form 3-dimensional tubular structures (Corada M, et al., Blood. 2002, 100:905-911). A modulating agent may inhibit the formation of endothelial tubes, and the cells may remain isolated or form clumps in the collagen.

Modulating Agent Modification and Formulations

A modulating agent as described herein may, but need not, be linked to one or more additional molecules. In particular, as discussed below, it may be beneficial for certain applications to link multiple modulating agents (which may, but need not, be identical) to a support material, such as a single molecule (e.g., keyhole limpet hemocyanin) or a solid support, such as a polymeric matrix (which may be formulated as a membrane or microstructure, such as an ultra thin film), a container surface (e.g., the surface of a tissue culture plate or the interior surface of a bioreactor), or a bead or other particle, which may be prepared from a variety of materials including glass, plastic or ceramics. For certain applications, biodegradable support materials are preferred, such as cellulose and derivatives thereof, collagen, spider silk or any of a variety of polyesters (e.g., those derived from hydroxy acids and/or lactones) or sutures (see U.S. Pat. No. 5,245,012). Within certain embodiments, modulating agents and molecules comprising other CAR sequence(s) (e.g., an HAV or RGD sequence) may be attached to a support such as a polymeric matrix, preferably in an alternating pattern.

Suitable methods for linking a modulating agent to a support material will depend upon the composition of the support and the intended use, and will be readily apparent to those of ordinary skill in the art. Attachment may generally be achieved through noncovalent association, such as adsorption or affinity or, preferably, via covalent attachment (which may be a direct linkage between a modulating agent and functional groups on the support, or may be a linkage by way of a cross-linking agent). Attachment of a modulating agent by adsorption may be achieved by contact, in a suitable buffer, with a solid support for a suitable amount of time. The contact time varies with temperature, but is generally between about 5 seconds and 1 day, and typically between about 10 seconds and 1 hour.

Covalent attachment of a modulating agent to a molecule or solid support may generally be achieved by first reacting the support material with a bifunctional reagent that will also react with a functional group, such as a hydroxyl or amino group, on the modulating agent. For example, a modulating agent may be bound to an appropriate polymeric support or coating using benzoquinone, by condensation of an aldehyde group on the support with an amine and an active hydrogen on the modulating agent or by condensation of an amino group on the support with a carboxylic acid on the modulating agent. A preferred method of generating a linkage is via amino groups using glutaraldehyde. A modulating agent may be linked to cellulose via ester linkages. Similarly, amide linkages may be suitable for linkage to other molecules such as keyhole limpet hemocyanin or other support materials. Multiple modulating agents and/or molecules comprising other CAR sequences may be attached, for example, by random coupling, in which equimolar amounts of such molecules are mixed with a matrix support and allowed to couple at random.

Although modulating agents as described herein may preferentially bind to specific tissues or cells, and thus may be sufficient to target a desired site in vivo, it may be beneficial for certain applications to include an additional targeting agent. Accordingly, a targeting agent may also, or alternatively, be linked to a modulating agent to facilitate targeting to one or more specific tissues. As used herein, a “targeting agent,” may be any substance (such as a compound or cell) that, when linked to a modulating agent enhances the transport of the modulating agent to a target tissue, thereby increasing the local concentration of the modulating agent. Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. Among the many monoclonal antibodies that may serve as targeting agents are anti-TAC, or other interleukin-2 receptor antibodies; 9.2.27 and NR-ML-05, reactive with the 250 kilodalton human melanoma-associated proteoglycan; and NR-LU-10, reactive with a pancarcinoma glycoprotein. An antibody targeting agent may be an intact (whole) molecule, a fragment thereof, or a functional equivalent thereof. Examples of antibody fragments are F(ab′)2, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers.

For certain embodiments, it may be beneficial to also, or alternatively, link a drug to a modulating agent. As used herein, the term “drug” refers to any bioactive agent intended for administration to a mammal to prevent or treat a disease or other undesirable condition. Drugs include hormones, growth factors, proteins, peptides and other compounds. The use of certain specific drugs within the context of the present invention is discussed below.

Modulating agents as described herein may be present within a pharmaceutical composition. A pharmaceutical composition comprises one or more modulating agents in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. One or more modulating agents (alone or in combination with a targeting agent and/or drug) may, but need not, be encapsulated within liposomes using well known technology. Compositions of the present invention may be formulated for any appropriate manner of administration, including for example, topical, oral, nasal, intravenous, intracranial, intraperitoneal, subcutaneous, or intramuscular administration.

For certain embodiments, as discussed herein, a pharmaceutical composition may further comprise a modulator of cell adhesion that is mediated by one or more molecules other than the particular VE-cadherin. Such modulators may generally be prepared as described above, using one or more CAR sequences and/or antibodies thereto. Such compositions are particularly useful for situations in which it is desirable to inhibit cell adhesion mediated by multiple cell adhesion molecules, such as other members of the cadherin gene superfamily such as the classical cadherins (e.g., N-cadherin and E-cadherin); nonclassical cadherins (e.g., OB-cadherin, cadherin-6, etc.), integrins; occludin; claudins; and/or extracellular matrix proteins such as laminin, fibronectin, collagens, vitronectin, entactin and tenascin, or members of the immunoglobulin superfamily (CEA, PE-CAM, N-CAM, L1 or JAM).

A pharmaceutical composition may also, or alternatively, contain one or more drugs, which may be linked to a modulating agent or may be free within the composition. Virtually any drug may be administered in combination with a modulating agent as described herein, for a variety of purposes as described below. Examples of types of drugs that may be administered with a modulating agent include analgesics, anesthetics, antianginals, antifungals, antibiotics, anticancer drugs (e.g., taxol or mitomycin C), antiinflammatories (e.g., ibuprofen and indomethacin), anthelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signaling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympathomimetics, sedatives, stimulants, sympathomimetics, toxins (e.g., cholera toxin), tranquilizers and urinary antiinfectives.

For imaging purposes, any of a variety of diagnostic agents may be incorporated into a pharmaceutical composition, either linked to a modulating agent or free within the composition. Diagnostic agents include any substance administered to illuminate a physiological function within a patient, while leaving other physiological functions generally unaffected. Diagnostic agents include metals, radioactive isotopes and radioopaque agents (e.g., gallium, technetium, indium, strontium, iodine, barium, bromine and phosphorus-containing compounds), radiolucent agents, contrast agents, dyes (e.g., fluorescent dyes and chromophores) and enzymes that catalyze a colorimetric or fluorometric reaction. In general, such agents may be attached using a variety of techniques as described above, and may be present in any orientation.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Sustained-release formulations may contain a modulating agent dispersed in a carrier matrix and/or contained within a reservoir surrounded by a rate controlling membrane (see, e.g., European Patent Application 710,491 A). Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Pharmaceutical compositions of the present invention may be administered in a manner appropriate to the disease to be treated (or prevented). Appropriate dosages and a suitable duration and frequency of administration will be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a modulating agent or pharmaceutical composition as described herein may be administered at a dosage ranging from 0.001 to 50 mg/kg body weight, preferably from 0.1 to 20 mg/kg, on a regimen of single or multiple daily doses. For topical administration, a cream typically comprises an amount of modulating agent ranging from 0.00001% to 1%, preferably 0.0001% to 0.002%. Fluid compositions typically contain about 10 ng/ml to 5 mg/ml, preferably from about 10 μg to 2 mg/mL modulating agent. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Modulating Agent Methods of Use

In general, the modulating agents and compositions described herein may be used for modulating a function of VE-cadherin-expressing cells such as cell adhesion, angiogenesis, maintenance of vascular integrity or regulation of vascular permeability. Such modulation may be performed in vitro and/or in vivo, preferably in a mammal such as a human, using any method that contacts the VE-cadherin-expressing cell with the modulating agent. As noted above, modulating agents for purposes that involve the disruption of VE-cadherin-mediated cell adhesion may comprise a VE-cadherin CAR sequence, multiple VE-cadherin CAR sequences in close proximity and/or a substance (such as an antibody or an antigen-binding fragment thereof) that recognizes a VE-cadherin CAR sequence. When it is desirable to also disrupt cell adhesion mediated by other adhesion molecules, a modulating agent may additionally comprise one or more CAR sequences bound by such adhesion molecules (and/or antibodies or fragments thereof that bind such sequences), preferably separated from each other and from the VE-cadherin CAR sequence by linkers. As noted above, such linkers may or may not comprise one or more amino acids.

For enhancing cell adhesion, as discussed above, a modulating agent may contain multiple VE-cadherin CAR sequences derived from either a particular VE-cadherin or antibodies (or fragments), preferably separated by linkers, and/or may be linked to a single molecule or to a support material. When it is desirable to also enhance cell adhesion mediated by other adhesion molecules, a modulating agent may additionally comprise one or more CAR sequences bound by such adhesion molecules (and/or antibodies or fragments thereof that bind such sequences), preferably separated from each other and from the VE-cadherin CAR sequence by linker.

Certain methods herein have an advantage over prior techniques in that they block or inhibit cell adhesion. As described in greater detail below, modulating agents as described herein may also be used to disrupt or enhance cell adhesion in a variety of other contexts. Within each of the methods described herein, one or more modulating agents may generally be administered alone, or within a pharmaceutical composition. In each specific method described herein, as noted above, a targeting agent may be employed to increase the local concentration of modulating agent at the target site.

Within one aspect, methods are provided in which cell adhesion is diminished. In one such aspect, the present invention provides methods for reducing unwanted cellular adhesion in a mammal by administering a modulating agent as described herein. Unwanted cellular adhesion can occur, for example, between endothelial cells, between tumor cells, between tumor cells and normal cells or between normal cells as a result of surgery, injury, chemotherapy, disease, inflammation, or other condition jeopardizing cell viability or function. Certain preferred modulating agents for use within such methods comprise one or more of the VE-cadherin CAR sequences provided herein. In one particularly preferred embodiment, a modulating agent is further capable of disrupting cell adhesion mediated by multiple adhesion molecules. Such an agent may comprise, in addition to one or more VE-cadherin CAR sequences, CAR sequences derived from other cell adhesion molecules, as discussed elsewhere herein, preferably separated from the VE-cadherin CAR sequence via a linker. Alternatively, separate modulators of cell adhesion mediated by other adhesion molecules may be administered in conjunction with the modulating agent(s), either within the same pharmaceutical composition or separately.

Topical administration of the modulating agent(s) is generally preferred, but other means may also be employed. Preferably, a fluid composition for topical administration (comprising, for example, physiological saline) comprises an amount of modulating agent as described above, and more preferably from 10 μg/mL to 1 mg/mL. Creams may generally be formulated as described above. Topical administration in the surgical field may be given once at the end of surgery by irrigation of the wound or as an intermittent or continuous irrigation with the use of surgical drains in the post-operative period or by the use of drains specifically inserted in an area of inflammation, injury or disease in cases where surgery does not need to be performed. Alternatively, parenteral or transcutaneous administration may be used to achieve similar results.

Within a related aspect, modulating agents as described herein may be used to increase the permeability of endothelial cell layers, thereby facilitating sampling of the blood compartment by passive diffusion. Such methods permit the detection and/or measurement of the levels of specific molecules circulating in the blood. In general, to sample the blood compartment, it is necessary to perturb adhesion between the endothelial cells of the microvasculature. Using currently available techniques, only small, uncharged molecules may be detected across skin in vivo. The methods described herein are not subject to the same degree of limitation. Accordingly, a wide variety of blood components may be sampled across endothelial cell layers. Such sampling may be achieved across any such cell layers, including skin and gums.

Within a further aspect, methods are provided for enhancing delivery of a drug to a tumor in a mammal, comprising administering a VE-cadherin modulating agent in combination with a drug to a tumor-bearing mammal. A modulating agent may further contain a CAR sequence derived from another cell adhesion molecule, such as an E- and/or N-cadherin CAR sequence (e.g., HAV, HAVD (SEQ ID NO: 113), SHAVSS (SEQ ID NO: 114), AHAVDI (SEQ ID NO: 115) or a analogue of such a sequence). Bi-functional modulating agents that comprise the VE-cadherin CAR sequence with either flanking E-cadherin-specific sequences or flanking N-cadherin-specific sequences joined via a linker to the VE-cadherin CAR sequence are also preferred. Preferably, the peptide portion(s) of a modulating agent comprises 6-16 amino acids, since longer peptides may be difficult to dissolve in aqueous solution and are more likely to be degraded by peptidases.

In one particularly preferred embodiment, a modulating agent is capable of disrupting cell adhesion mediated by multiple adhesion molecules. For example, a single branched modulating agent (or multiple agents linked to a single molecule or support material) may disrupt adhesion mediated by a VE-cadherin, as well as E-cadherin, N-cadherin, OB-cadherin, occludin, claudin and/or integrin mediated cell adhesion. Such agents serve as multifunctional disrupters of cell adhesion. Alternatively, a separate modulator may be administered in conjunction with the modulating agent(s), either within the same pharmaceutical composition or separately. Preferred antibody modulating agents include Fab fragments directed against a nonclassical or classical cadherin CAR sequence, as described above. A Fab fragment may be incorporated into a modulating agent or may be present within a separate modulator that is administered concurrently.

Preferably, the modulating agent and the drug are formulated within the same composition or drug delivery device prior to administration. In general, a modulating agent may enhance drug delivery to any tumor (e.g., breast tumor, stomach tumor, ovarian tumor, brain tumor or kidney tumor, and the method of administration may be chosen based on the type of target tumor. For example, injection or topical administration. as described above may be preferred for melanomas and other accessible tumors (e.g., metastases from primary ovarian tumors may be treated by flushing the peritoneal cavity with the composition). Other tumors (e.g., breast tumors) may be treated by injection of the modulating agent and the drug (such as mitomycin C) into the site of the tumor. In other instances, the composition may be administered systemically, and targeted to the tumor using any of a variety of specific targeting agents. Suitable drugs may be identified by those of ordinary skill in the art based upon the type of cancer to be treated (e.g., taxol for breast cancer). In general, the amount of modulating agent administered varies with the method of administration and the nature of the tumor, within the typical ranges provided above, preferably ranging from about 1 μg/mL to about 2 mg/mL, and more preferably from about 10 μg/mL to 1 mg/mL. Transfer of the drug to the target tumor may be evaluated by appropriate means that will be apparent to those of ordinary skill in the art. Drugs may also be labeled (e.g., using radionuclides) to permit direct observation of transfer to the target tumor using standard imaging techniques.

Within certain preferred aspects, the present invention provides methods for treating cancer and metastasis by administering to a mammal one or more modulating agents of the present invention. The cancer may be essentially any cancer type which expresses VE-cadherin and/or which requires a blood supply for its growth or survival. Cancers that may express VE-cadherin include, for example, hemangiomas, hemangioendotheliomas, angiosarcomas, Kaposi's sarcoma and epitheloid sarcomas. Cancers which require a blood supply include all tumors that grow beyond the limits of diffusion of nutrients (Folkman, Semin Oncol. 2002 December;29(6 Suppl 16):15-8. ). As VE-cadherin is involved in angiogenesis and maintenance of vascular integrity, cancer types which may be treated with VE-cadherin modulating agents include all those which rely upon a blood supply for their growth or survival, including for example those which are highly vascularized, such as, renal adenocarcinomas and glioblastomas.

A modulating agent may be administered alone (e.g., via the skin) or within a pharmaceutical composition. For accessible tumors, injection or topical administration as described above may be preferred. In other instances, the composition may be administered systemically, and targeted to the tumor using any of a variety of specific targeting agents, as described above. In general, the amount of agent administered varies depending upon the method of administration and the nature of the cancer, but may vary within the ranges identified above. The effectiveness of the cancer treatment or inhibition of metastasis may be evaluated using well known clinical observations, such as monitoring the level of serum tumor markers (e.g., CEA or PSA).

The addition of a targeting agent as described above may be beneficial, particularly when the administration is systemic. Suitable modes of administration and dosages depend upon the condition to be prevented or treated but, in general, administration by injection is appropriate. Dosages may vary as described above. The effectiveness of the inhibition may be evaluated grossly by assessing the inability of the tumors to maintain their growth and microscopically by observing an absence of nerves at the periphery of the tumor.

Within further aspects, the present invention provides methods for inhibiting angiogenesis (i.e., the growth of blood vessels from pre-existing blood vessels) in a mammal. Inhibition of angiogenesis may be beneficial, for example, in patients afflicted with diseases such as cancer, obesity or arthritis. Preferred modulating agents for inhibition of angiogenesis include those that modulate functions mediated by VE-cadherins. In addition, a modulating agent for use in inhibiting angiogenesis may further comprise a separate CAR sequence from a different cell adhesion molecule, as discussed above, such as the sequence RGD, which is recognized by integrins, the classical cadherin CAR sequence HAV, and/or the occludin CAR sequence LYHY (SEQ ID NO:105), separated from the VE-cadherin CAR sequence via a linker. Alternatively, a separate modulator of classical cadherin-, integrin- or occludin-mediated cell adhesion may be administered in conjunction with the modulating agent(s), either within the same pharmaceutical composition or separately. The ability of a modulating agent to inhibit angiogenesis may be evaluated as described above.

The addition of a targeting agent as described above may be beneficial, particularly when the administration is systemic. Suitable modes of administration and dosages depend upon the condition to be prevented or treated but, in general, administration by injection is appropriate. Dosages may vary as described above. The effectiveness of the inhibition may be evaluated in the case of cancer grossly by assessing the inability of the tumors to maintain their growth and microscopically by observing an absence of nerves at the periphery of the tumor.

In yet another related aspect, the present invention provides methods for modulating cell survival, such as methods for inducing apoptosis in a cadherin-expressing cell. In general, patients afflicted with cancer may benefit from such treatment. Modulating agents for use within such methods may modulate functions mediated VE-cadherin and/or other classical and nonclassical cadherin(s). Such agents comprise a VE-cadherin CAR sequence, and may further comprise, for example, a CAR sequence of a different cell adhesion molecule, as discussed above, or an-analogue of such a sequence. In one embodiment, the peptide portion(s) of such modulating agents comprise 6-16 amino acids, however it will be appreciated that both shorter and longer modulating agents may also be used. Preferred antibody modulating agents in this context include Fab fragments directed against VE-cadherin and/or a nonclassical or classical cadherin CAR sequence. The Fab fragments may be either incorporated into a modulating agent or within a separate modulator that is administered concurrently. Administration may be topical, via injection or by other means, and the addition of a targeting agent may be beneficial, particularly when the administration is systemic. Suitable modes of administration and dosages depend upon the location and nature of the cells for which induction of apoptosis is desired but, in general, dosages may vary as described above. A biopsy may be performed to evaluate the level of induction of apoptosis.

In another embodiment, methods are provided for causing the regression of blood vessels for the treatment of conditions such as cancer, psoriasis, arthritis, obesity and age-related macular degeneration. Cancer tumors are solid masses of cells, growing out of control, which require nourishment via blood vessels. The formation of new capillaries is a prerequisite for tumor growth and the emergence of metastases. Administration of the modulating agents described herein may disrupt blood vessels and cause them to regress, thereby providing effective therapy for patients afflicted with diseases such as cancer. Certain preferred modulating agents for use within such methods comprise, in addition to a VE-cadherin CAR sequence, a separate CAR sequence from a different cell adhesion molecule, as described above, such as HAV and RGD, or an analogue of such a sequence. Preferably, the peptide portion(s) of such modulating agents comprise 6-16 amino acids. Preferred antibody modulating agents include Fab fragments directed against the VE-cadherin CAR sequence, with or without Fab fragments directed against one or more other cadherin CAR sequences. The Fab fragments may be either incorporated into a modulating agent or within a separate modulator that is administered concurrently. Administration may be topical, via injection or by other means, and the addition of a targeting agent may be beneficial, particularly when the administration is systemic. Suitable modes of administration and dosages depend upon the location and nature of the vasculature for which disruption of cell adhesion is desired but, in general, dosages may vary as described above. The effectiveness of the cancer treatment or inhibition of metastasis may be evaluated using well known clinical observations such as the level of serum markers (e.g., CEA or PSA). The addition of a targeting agent may be beneficial, particularly when the administration is systemic. Suitable modes of administration and dosages depend upon the condition to be prevented or treated but, in general, administration by injection is appropriate. Dosages may vary as described above. The effectiveness of the inhibition may be evaluated grossly by assessing the inability of the tumor to maintain growth and microscopically by an absence of nerves at the periphery of the tumor.

Within another aspect, the present invention provides methods for enhancing drug delivery to the central nervous system (CNS) of a mammal. The blood/brain barrier is largely impermeable to most neuroactive agents, and delivery of drugs to the brain of a mammal often requires invasive procedures. Using a modulating agent as described herein, however, delivery may be by, for example, systemic administration of a modulating agent-drug-targeting agent combination, injection of a modulating agent (alone or in combination with a drug and/or targeting agent) into the carotid artery or application of a skin patch comprising a modulating agent to the head of the patient. Modulating agents for enhancing drug delivery to the central nervous system include those agents that disrupt functions mediated by VE-cadherin. Certain preferred modulating agents for use within such methods are relatively small cyclic peptides (e.g., a ring size of 4-10 residues; preferably 5-7 residues). Also preferred are multi-functional modulating agents comprising a VE-cadherin CAR and further comprising a separate CAR sequence from another cell adhesion molecule, as described above, such as an N-cadherin CAR sequence, the claudin CAR sequence IYSY (SEQ ID NO:108) and/or occludin CAR sequence, preferably joined by a linker. Alternatively, a separate modulator of N-cadherin, claudin and/or occludin-mediated cell adhesion may be administered in conjunction with the VE-cadherin modulating agent(s), either within the same pharmaceutical composition or separately. Modulating agents may further comprise antibodies or Fab fragments directed, for example, against the N-cadherin CAR sequence FHLRAHAVDINGNQV-NH2 (SEQ ID NO: 116). Fab fragments directed against the occludin CAR sequence GVNPTAQSSGSLYGSQIYALCNQFYTPAATGLYVDQYLYHYCVVDPQE (SEQ ID NO: 117) may also be employed, either incorporated into the modulating agent or administered concurrently as a separate modulator. In general, the amount of modulating agent administered varies with the method of administration and the nature of the condition to be treated or prevented, but typically varies as described above. Transfer of the drug to the central nervous system may be evaluated by appropriate means that will be apparent to those of ordinary skill in the art, such as magnetic resonance imaging (MRI) or PET scan (positron emitted tomography).

The present invention also provides methods for increasing vasopermeability in a mammal by administering one or more VE-cadherin modulating agents or pharmaceutical compositions. Such agents may comprise, in addition to a VE-cadherin CAR sequence, a CAR sequence from another cell adhesion molecule, as discussed above, such as LYHY (the occludin CAR sequence; SEQ ID NO:105), IYSY (the claudin CAR sequence; SEQ ID NO:108) HAV and RGD, or an analogue of such a sequence. Preferably, the peptide portion(s) of such modulating agents comprise 6-16 amino acids. Preferred antibody modulating agents include Fab fragments directed against VE-cadherin, and may further comprise Fab fragments directed against other CAR sequences such as one or more CAR sequences from OB-cadherin, classical cadherins, claudins and/or occluding. The Fab fragments may be either incorporated into a modulating agent or within a separate modulator that is administered concurrently.

Treatment with a modulating agent may be appropriate, for example, prior to administration of an anti-tumor therapeutic or diagnostic agent (e.g., a monoclonal antibody or other macromolecule), an antimicrobial agent or an anti-inflammatory agent, in order to increase the concentration of such agents in the vicinity of the target tumor, organism or inflammation without increasing the overall dose to the patient. Modulating agents for use within such methods may be linked to a targeting agent to further increase the local concentration of modulating agent, although systemic administration of a vasoactive agent even in the absence of a targeting agent increases the perfusion of certain tumors relative to other tissues. Suitable targeting agents include antibodies and other molecules that specifically bind to tumor cells or to components of structurally abnormal blood vessels. For example, a targeting agent may be an antibody that binds to a fibrin degradation product or a cell enzyme such as a peroxidase that is released by granulocytes or other cells in necrotic or inflamed tissues.

Administration via intravenous injection or transdermal administration is generally preferred. Effective dosages are generally sufficient to increase localization of a subsequently administered diagnostic or therapeutic agent to an extent that improves the clinical efficacy of therapy of accuracy of diagnosis to a statistically significant degree. Comparison may be made between treated and untreated tumor host animals to whom equivalent doses of the diagnostic or therapeutic agent are administered. In general, dosages range as described above.

In certain other aspects, the present invention provides methods for enhancing adhesion of VE-cadherin-expressing cells. Within certain embodiments, a modulating agent may be linked to a solid support, resulting in a matrix that comprises multiple modulating agents. Within one such embodiment, the support is a polymeric matrix to which modulating agents and molecules comprising other CAR sequence(s) are attached (e.g., modulating agents and molecules comprising either HAV or RGD sequences may be attached to the same matrix, preferably in an alternating pattern). Such matrices may be used in contexts in which it is desirable to enhance adhesion mediated by multiple cell adhesion molecules. Alternatively, the modulating agent itself may comprise multiple VE-cadherin CAR sequences or antibodies (or fragments thereof), separated by linkers as described above. Either way, the modulating agent(s) function as a “biological glue” to bind multiple nonclassical cadherin-expressing cells within a variety of contexts.

Within one such aspect, modulating agents comprising a VE-cadherin CAR sequence and/or multiple modulating agents linked to a single molecule or support material may be used to facilitate wound healing and/or reduce scar tissue in a mammal. The modulating agents may further comprise CAR sequences from other cell adhesion molecules, as described herein, such as desmoglein and/or desmocollin CAR sequences. Additionally, other CAR sequences include HAV, HAVD (SEQ ID NO: 113), SHAVSS (SEQ ID NO:114), AHAVDI (SEQ ID NO:115), or an analogue of such a sequence. Preferred antibody modulating agents include Fab fragments directed against either the VE-cadherin CAR sequence and may further comprise Fab fragments directed against nonclassical cadherin, N-cadherin or E-cadherin CAR sequences. Modulating agents that are linked to a biocompatible and biodegradable matrix such as cellulose or collagen are particularly preferred. For use within such methods, a modulating agent should have a free amino or hydroxyl group. The modulating agents are generally administered topically to the wound, where they may facilitate closure of the wound and may augment, or even replace, stitches. Similarly, administration of matrix-linked modulating agents may facilitate cell adhesion in skin grafting and prosthetic implants, and may prolong the duration and usefulness of collagen injection. In general, the amount of matrix-linked modulating agent administered to a wound, graft or implant site varies with the severity of the wound and/or the nature of the wound, graft, or implant, but may vary as discussed above. Multi-functional modulating agents comprising a VE-cadherin CAR sequence and further comprising one or more CAR sequences from another cell adhesion molecules, as described herein, such as a nonclassical cadlerin CAR sequence, a classical cadherin CAR sequence (HAV), and/or the CAR sequence bound by certain integrins (RGD) may also be used as potent stimulators of wound healing and/or to reduce scar tissue. Alternatively, one or more separate modulators of classical cadherin- or integrin-mediated cell adhesion may be administered in conjunction with the modulating agent(s), either within the same pharmaceutical composition or separately.

Within another aspect, one or more modulating agents may be linked to the interior surface of a tissue culture plate or other cell culture support, such as for use in a bioreactor. Such linkage may be performed by any suitable technique, as described above. Modulating agents linked in this fashion may generally be used to immobilize cadherin-expressing cells. For example, dishes or plates coated with one or more modulating agents may be used to immobilize cadherin-expressing cells within a variety of assays and screens. Within bioreactors (i.e., systems for large scale production of cells or organoids), modulating agents may generally be used to improve cell attachment and stabilize cell growth. Modulating agents may also be used within bioreactors to support the formation and function of highly differentiated organoids derived, for example, from dispersed populations of fetal mammalian cells. Bioreactors containing biomatrices of modulating agent(s) may also be used to facilitate the production of specific proteins.

Modulating agents as described herein may be used within a variety of bioreactor configurations. In general, a bioreactor is designed with an interior surface area sufficient to support large numbers of adherent cells. This surface area can be provided using membranes, tubes, microtiter wells, columns, hollow fibers, roller bottles, plates, dishes, beads or a combination thereof. A bioreactor may be compartmentalized. The support material within a bioreactor may be any suitable material known in the art; preferably, the support material does not dissolve or swell in water. Preferred support materials include, but are not limited to, synthetic polymers such as acrylics, vinyls, polyethylene, polypropylene, polytetrafluoroethylene, nylons, polyurethanes, polyamides, polysulfones and poly(ethylene terephthalate); ceramics; glass and silica.

Within further aspects, modulating agents as described herein may be used for modulating the immune system of a mammal in any of several ways. Cadherins are expressed on immature B and T cells (thymocytes and bone marrow pre-B cells), as well as on specific subsets of activated B and T lymphocytes and some hematological malignancies. Modulating agents may generally be used to modulate specific steps within cellular interactions during an immune response or during the dissemination of malignant lymphocytes.

For example, a modulating agent as described herein may be used to treat diseases associated with excessive generation of otherwise normal T cells. Without wishing to be bound by any particular theory, it is believed that the interaction of cadherins on maturing T cells and B cell subsets contributes to protection of these cells from programmed cell death. A modulating agent may decrease such interactions, leading to the induction of programmed cell death. Accordingly, modulating agents may be used to treat certain types of diabetes and rheumatoid arthritis, particularly in young children where the cadherin expression on thymic pre-T cells is greatest.

Modulating agents may also be administered to patients afflicted with certain skin disorders (such as cutaneous lymphomas), acute B cell leukemia and excessive immune reactions involving the humoral immune system and generation of immunoglobulins, such as allergic responses and antibody-mediated graft rejection. In addition, patients with circulating cadherin-positive malignant cells (e.g., during regimes where chemotherapy or radiation therapy is eliminating a major portion of the malignant cells in bone marrow and other lymphoid tissue) may benefit from treatment with a modulating agent. Such treatment may also benefit patients undergoing transplantation with peripheral blood stem cells.

Preferred modulating agents for use within such methods include those that disrupt VE-cadherin, as described herein. The modulating agents may additionally contain CAR sequence designed to disrupt cadherin-6 and/or cadherin-8 mediated cell adhesion, for example. In addition, other illustrative modulating agents may comprise one or more additional CAR sequences, as described herein, such as HAV, RGD, LYHY (SEQ ID NO:105) and/or KYSFNYDGSE (SEQ ID NO:103). As noted above, such additional sequence(s) may be separated from a nonclassical CAR sequence via a linker. Alternatively, a separate modulator of classical cadherin-, occludin-, integrin- and/or N-CAM-mediated cell adhesion may be administered in conjunction with the modulating agent(s), either within the same pharmaceutical composition or separately.

Within the above methods, the modulating agent(s) are, preferably administered systemically (usually by injection) or topically. A modulating agent may be linked to a targeting agent. For example, targeting to the bone marrow may be beneficial. A suitable dosage is sufficient to effect a statistically significant reduction in the population of B and/or T cells that express cadherin and/or an improvement in the clinical manifestation of the disease being treated. Typical dosages generally range as described above.

Within further aspects, the present invention provides methods and kits for preventing pregnancy in a mammal. For example, disruption of VE-cadherin function prevents or inhibits angiogenesis, a process required for placenta formation. In one embodiment, one or more modulating agents may be incorporated into any of a variety of well known contraceptive devices, such as sponges suitable for intravaginal insertion (see, e.g., U.S. Pat. No. 5,417,224) or capsules for subdermal implantation. Other modes of administration are possible, however, including transdermal administration, for modulating agents linked to an appropriate targeting agent. Preferred modulating agents for use within such methods include those comprising a VE-cadherin CAR sequence, as described herein, and may further comprise, for example, one or more CAR sequences from a different cell adhesion molecule, as described herein, such as an OB-cadherin CAR sequence, or analogue or mimetic thereof. In addition, other illustrative modulating agents may comprise additional CAR sequences, such as HAV and/or RGD. As noted above, such additional sequences may be separated from the nonclassical CAR sequence via a linker. Alternatively, a separate modulator of classical cadherin- and/or integrin-mediated cell adhesion may be administered in conjunction with the VE-cadherin modulating agent(s), either within the same pharmaceutical composition or separately.

Suitable methods for incorporation into a contraceptive device depend upon the type of device and are well known in the art. Such devices facilitate administration of the modulating agent(s) to the uterine region and may provide a sustained release of the modulating agent(s). In general, modulating agent(s) may be administered via such a contraceptive device at a dosage ranging from 0.1 to 50 mg/kg, although appropriate dosages may be determined by monitoring hCG levels in the urine. hCG is produced by the placenta, and levels of this hormone rise in the urine of pregnant women. The urine hCG levels can be assessed by radio-immunoassay using well known techniques. Kits for preventing pregnancy generally comprise a contraceptive device impregnated with one or more modulating agents.

Alternatively, a sustained release formulation of one or more modulating agents may be implanted, typically subdermally, in a mammal for the prevention of pregnancy. Such implantation may be performed using well known techniques. Preferably, the implanted formulation provides a dosage as described above, although the minimum effective dosage may be determined by those of ordinary skill in the art using, for example, an evaluation of hCG levels in the urine of women.

Other aspects of the present invention provide methods that employ antibodies raised against VE-cadherin CAR sequences for diagnostic and assay purposes. Assays typically involve using an antibody to detect the presence or absence of a VE-cadherin sequence (free or on the surface of a cell), or proteolytic fragments containing one or more EC domains in a suitable biological sample, such as tumor or normal tissue biopsies, blood, lymph node, serum or urine samples, or other tissue, homogenate, or extract thereof obtained from a patient.

There are a variety of assay formats known to those of ordinary skill in the art for using an antibody to detect a target molecule in a sample. See, e.g., Harlow and Lane, Antibodies: A Laboratory Manual, Cold Spring Harbor Laboratory, 1988. For example, the assay may be performed in a Western blot format, wherein a protein preparation from the biological sample is submitted to gel electrophoresis, transferred to a suitable membrane and allowed to react with the antibody. The presence of the antibody on the membrane may then be detected using a suitable detection reagent, as described below.

In another embodiment, the assay involves the use of antibody immobilized on a solid support to bind to the target VE-cadherin, or a proteolytic fragment containing an extracellular domain and encompassing a CAR sequence, and remove it from the remainder of the sample. The bound cadherin may then be detected using a second antibody or reagent that contains a reporter group. Alternatively, a competitive assay may be utilized, in which a cadherin is labeled with a reporter group and allowed to bind to the immobilized antibody after incubation of the antibody with the sample. The extent to which components of the sample inhibit the binding of the labeled cadherin to the antibody is indicative of the reactivity of the sample with the immobilized antibody, and as a result, indicative of the level of the cadherin in the sample.

The solid support may be any material known to those of ordinary skill in the art to which the antibody may be attached, such as a test well in a microtiter plate, a nitrocellulose filter or another suitable membrane. Alternatively, the support may be a bead or disc, such as glass, fiberglass, latex or a plastic such as polystyrene or polyvinylchloride. The antibody may be immobilized on the solid support using a variety of techniques known to those in the art, which are amply described in the patent and scientific literature.

In certain embodiments, the assay for detection of a VE-cadherin in a sample is a two-antibody sandwich assay. This assay may be performed by first contacting -an antibody that has been immobilized on a solid support, commonly the well of a microtiter plate, with the biological sample, such that the VE-cadherin within the sample is allowed to bind to the immobilized antibody (a 30 minute incubation time at room temperature is generally sufficient). Unbound sample is then removed from the immobilized VE-cadherin-antibody complexes and a second antibody (containing a reporter group such as an enzyme, dye, radionuclide, luminescent group, fluorescent group or biotin) capable of binding to a different site on the VE-cadherin is added. The amount of second antibody that remains bound to the solid support is then determined using a method appropriate for the specific reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products. Standards and standard additions may be used to determine the level of cadherin in a sample, using well known techniques.

The present invention also provides kits for use in such immunoassays. Such kits generally comprise one or more antibodies, as described above. In addition, one or more additional compartments or containers of a kit generally enclose elements, such as reagents, buffers and/or wash solutions, to be used in the immunoassay.

Within further aspects, modulating agents. or antibodies (or fragments thereof) may be used to facilitate cell identification and sorting in vitro or imaging in vivo, permitting the selection of cells expressing VE-cadherin (or different VE-cadherin levels). Preferably, the modulating agent(s) or antibodies for use in such methods are linked to a detectable marker. Suitable markers are well known in the art and include radionuclides, luminescent groups, fluorescent groups, enzymes, dyes, constant immunoglobulin domains and biotin. Within one preferred embodiment, a modulating agent linked to a fluorescent marker, such as fluorescein, is contacted with the cells, which are then analyzed by fluorescence activated cell sorting (FACS).

Antibodies or fragments thereof may also be used within screens of combinatorial or other nonpeptide-based libraries to identify other compounds capable of modulating VE-cadherin-mediated cell adhesion. Such screens may generally be performed using an ELISA or other method well known to those of ordinary skill in the art that detect compounds with a shape and structure similar to that of the modulating agent. In general, such screens may involve contacting an expression library producing test. compounds with an antibody, and detecting the level of antibody bound to the candidate compounds. Compounds for which the antibody has a higher affinity may be further characterized as described herein, to evaluate the ability to modulate VE-cadherin-mediated functions.

Within other aspects, modulating agents of the invention may be used to remove metastatic cells from a biological sample, such as blood, bone marrow or a fraction thereof. Such removal may be achieved by contacting a biological sample with a modulating agent under conditions and for a time sufficient to permit VE-cadherin expressing cells to bind to the modulating agent. The VE-cadherin expressing cells that have bound to the modulating agent are then separated from the remainder of the sample. To facilitate this removal, a modulating agent may be linked to a solid support. Preferably, the contact results in the reduction of VE-cadherin expressing cells in the sample to less than 1%, preferably less than 0.1%, of the level prior to contact with the modulating agent. The extent to which such cells have been removed may be readily determined by standard methods such as, for example, qualitative and quantitative PCR analysis, immunohistochemistry and FACS analysis. Following removal of metastatic cells, the biological sample may be returned to the patient using standard techniques.

Within other aspects, the present invention provides compositions and methods for diagnosing a cancer, particularly a cancer that expresses VE-cadherin, or that is associated with cells that express VE-cadherin, such as breast, ovarian and prostate cancer, as well as leukemia. Certain methods provided herein employ binding agents, such as antibodies and fragments thereof, that specifically recognize VE -cadherin. Other methods employ one or more polynucleotides capable of hybridizing to a polynucleotide encoding VE-cadherin.

Within certain aspects, the present invention provides methods for determining the presence or absence of a cancer in a patient, comprising the steps of: (a) contacting a biological sample obtained from a patient with a binding agent that specifically binds to VE-cadherin; and (b) detecting in the sample an amount of polypeptide that binds to the binding agent, relative to a predetermined cut-off value, and therefrom determining the presence or absence of a cancer in the patient.

Within further aspects, methods are provided for monitoring the progression of a cancer in a patient, comprising the steps of: (a) contacting a biological sample obtained from a patient at a first point in time with a binding agent that specifically binds to VE-cadherin; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) to the amount detected in step (b) and therefrom monitoring the progression of the cancer in the patient.

Within other aspects, methods are provided for evaluating the metastatic potential of a cancer in a patient, comprising the steps of: (a) contacting a biological sample obtained from a patient afflicted with cancer with a binding agent that specifically binds to VE-cadherin; and (b) detecting in the sample an amount of polypeptide that binds to the binding agent, relative to a predetermined cut-off value, and therefrom evaluating the metastatic potential of the cancer in the patient. Kits for determining the presence or absence of a cancer in a patient are also provided. Such kits may comprise: (a) an antibody or antigen-binding fragment thereof that specifically binds to a VE-cadherin CAR sequence; and (b) a detection reagent.

The present invention further provides methods for determining the presence or absence of a metastatic cancer in a patient, comprising the steps of: (a) contacting a biological sample obtained from a patient with an oligonucIeotide that hybridizes to a polynucleotide encoding VE-cadherin; and (b) detecting in the sample a level of a polynucleotide that hybridizes to the oligonucleotide, relative to a predetermined cut-off value, and therefrom determining the presence or absence of a metastatic cancer in the patient. Within certain embodiments, the amount of mRNA is detected via polymerase chain reaction using, for example, at least one oligonucleotide primer that hybridizes to a polynucleotide that encodes VE-cadherin, or a complement of such a polynucleotide. Within other embodiments, the amount of mRNA is detected using a hybridization technique, employing an oligonucleotide probe that hybridizes to a polynucleotide that encodes VE-cadherin, or a complement of such a polynucleotide. In a preferred embodiment, at least one of the oligonucleotide primers comprises at least about 10 contiguous nucleotides of a DNA molecule encoding VE-cadherin.

In related aspects, methods are provided for monitoring progression of a cancer in a patient, comprising the steps of: (a) contacting a biological sample obtained from a patient with an oligonucleotide that hybridizes to a polynucleotide encoding VE-cadherin; (b) detecting in the sample an amount of polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring progression of a cancer in the patient.

Within other aspects, methods are provided for evaluating the metastatic potential of a cancer in a patient, comprising the steps of: (a) contacting a biological sample obtained from a patient with an oligonucleotide that hybridizes to a polynucleotide encoding VE-cadherin; and (b) detecting in the sample an amount of a polynucleotide that hybridizes to the oligonucleotide, relative to a predetermined cut-off value, and therefrom evaluating the metastatic potential of the cancer in the patient.

In related aspects, diagnostic kits comprising the above oligonucleotide probes or primers are provided. The following Examples are offered by way of illustration and not by way of limitation.

### EXAMPLES

### Example 1

### Preparation of Representative Modulating Agents

This Example illustrates the solid phase synthesis of representative peptide modulating agents.

The peptides were synthesized on a 431A Applied Biosystems peptide synthesizer using p-Hydroxymethylphenoxymethyl polystyrene (HMP) resin and standard Fmoc chemistry. After synthesis and deprotection, the peptides were de-salted on a Sephadex G-10 column and lyophilized. The peptides were analyzed for purity by analytical HPLC, and in each case a single peak was observed. Peptides were made as stock solutions at 10 to 25 mg/mL in dimethylsulfoxide (DMSO) or water and stored at −20° C. before use.

### Example 2

### Disruption of Endothelial Cell Adhesion Using VE-Cadherin Peptide Modulating Agents

This Example illustrates the ability of a representative linear peptide comprising a cadherin-5 CAR sequence to disrupt endothelial cell adhesion.

Human umbilical vein endothelial cells were cultured using standard procedures (see Ichikawa et al., Amer. J. Physiol. 273 (Gastrointest. Liver Physiol. 36):3642-6347, 1997). Cells were maintained in EGM (Clonetics, San Diego, Calif.) and used at P2 for all experiments. Endothelial identity was established by Dil-LDL and factor VIII staining.

The cells were cultured on glass coverslips. Monolayers were exposed to peptides at a concentration of 75 μg/mL for 60 minutes. The cells were then fixed with 95% ethanol for 30 minutes at 4° C., followed by acetone for one minute and left to air dry at room temperature. Primary antibody for VE-cadherin (Immunotech, Marseilles, France; 1:250) was added for one hour at 37° C. Coverslips were then washed with 0.1% milk/PBS solution three times for five minutes each. Secondary antibody (1:250), goat anti-rabbit FITC (Zymed, San Francisco, Calif.) was incubated at 37° C. for one hour. Coverslips were again washed with 0.1% milk/PBS solution three times for five minutes each. Coverslips were mounted with anti-quenching solution (1 mg/mL phenylenediamine (Sigma, St. Louis, Mo.) in 50% glycerol, 50% PBS). All photographs were taken at 400× and 1000× with exposure times of 12 seconds.

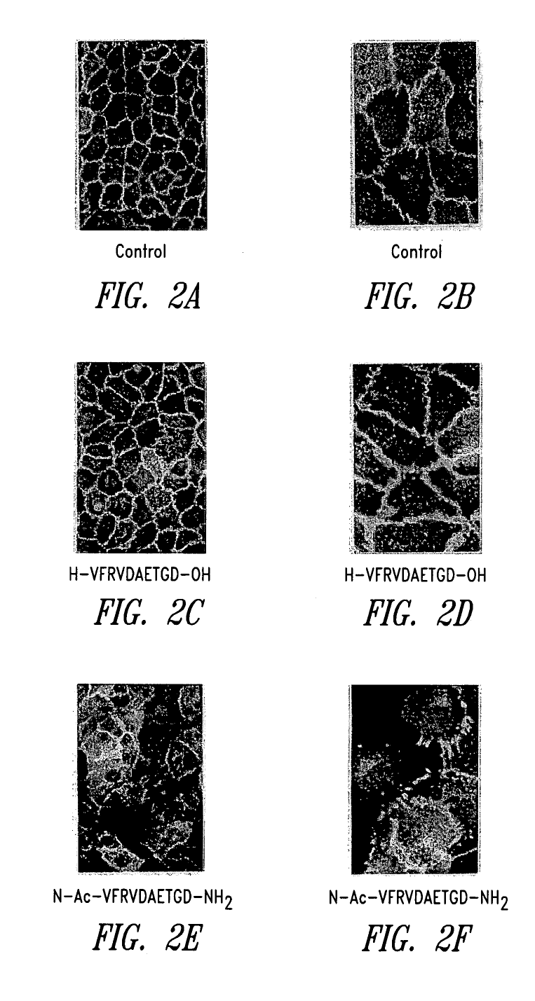

The resulting photographs are presented in FIGS. 2A-2F. FIGS. 2A and 2B are control cells. The cells in FIGS. 2C and 2D were exposed to 75 μg/mL of H-VFRVDAETGD-OH (SEQ ID NO: 19) and the cells in FIGS. 2E and 2F were exposed to 75 μg/mL of the linear peptide modulating agent N-Ac-VFRVDAETGD-NH₂ (SEQ ID NO: 19). These results indicate that the linear peptide modulating agent N-Ac-VFRVDAETGD-NH₂ (SEQ ID NO: 19) disrupts endothelial cell adhesion, with an activity that is substantially greater that that of a similar peptide without the N- and C-terminal functional groups.

### Example 3

### Inhibition of Endothelial Tube Formation Upon Treatment with VE-Cadherin Peptide Modulating Agents

Endothelial cells were harvested from human umbilical vein and cultured as previously described (Lampugnani et al., 1995 JCB 129 203-217). Briefly, human endothelial cells were cultured on 1% gelatin-coated flask in medium 199 with 20% newborn calf serum (NCS) supplemented with 50 ug/mL endothelial growth supplement (ECGS) and 100 ug/mL heparin. Collagen tube formation assays were performed as previously described (Corada et al 2002 Blood 100 905-911; Corada et al 2001 Blood 97 1679-1684). Briefly, type I collagen (Collaborative Biomedical Product, Bedford, Mass.) from rat tail was diluted to a concentration of 1 mg/mL, and pH was neutralized by adding 1/10 volume of 10× minimal essential medium (MEM) (Life Technologies). Aliquots of 250 μL were added to each well of a 24-well culture plates and incubated at 37° C. until gelation occurred. Human umbilical vein endothelial cells were seeded on the collagen gel at 10,000 cells/mL in complete medium in the presence of peptides or controls for 24 hr. Medium was then removed and a second collagen gel coat was made on top of the cells (in a sandwich fashion) by adding soluble collagen for 30 min at 37° C. After this period, complete medium was added containing peptides or controls. Cells were left for 24 hr to form tubes. Tube formation was assessed by phase contrast microscopy.

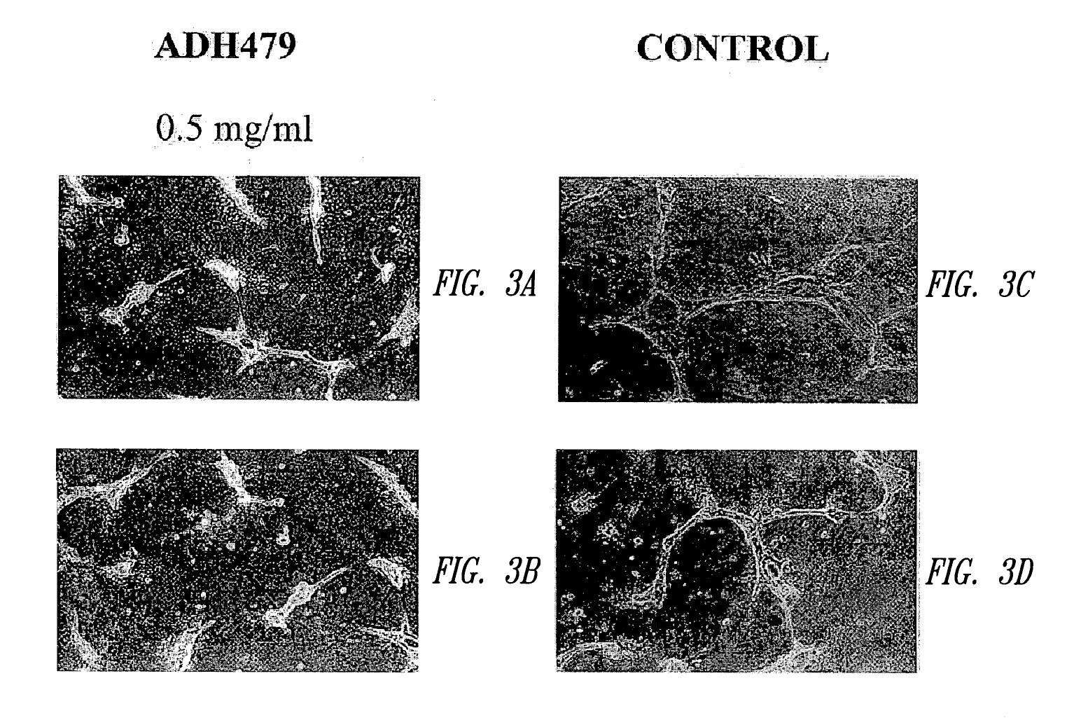

In the presence of control (sucrose carrier), endothelial cells formed tube-like structures within the collagen gel (FIGS. 3C and. 3D). In the presence of cadherin-modulating peptide ADH479 (Ac-FRVDAETGDVFAIER-NH₂; SEQ ID NO: 18) at 0.5 mg/mL, significant inhibition of endothelial tube formation was observed (FIGS. 3A and 3B).

### Example 4

### Increased Migration of Endothelial Cells Upon Treatment with VE-Cadherin Peptide Modulating Agents

Endothelial cells were harvested from human umbilical vein and cultured as previously described (Lampugnani et al., 1995 JCB 129 203-217). Briefly, human endothelial cells are cultured on 1% gelatin-coated flask in medium 199 with 20% newborn calf serum (NCS) supplemented with 50 ug/mL endothelial growth supplement (ECGS) and 100 ug/mL heparin.

In vitro wounding for testing cell migration was performed following a previously published procedure (Lampugnani et al., 1995 JCB 129 203-217; Brevario et al., 1995 Atherosclerosis Thromb Vasc Biol 15 1229-1239; Navarro et al., 1995 JCB 270 30965-30972). Endothelial cells were cultured for 5 days in 24-well plates on gelatin to obtain a tightly confluent monolayer. Culture medium was then aspirated, and the cell monolayer was wounded with a plastic tip. The wounded cell layer was washed twice with culture medium and incubated with complete medium in the presence or absence of VE-cadherin-modulating peptides. After 20 hr of migration the cells were fixed with Fast Green and stained with crystal violet.

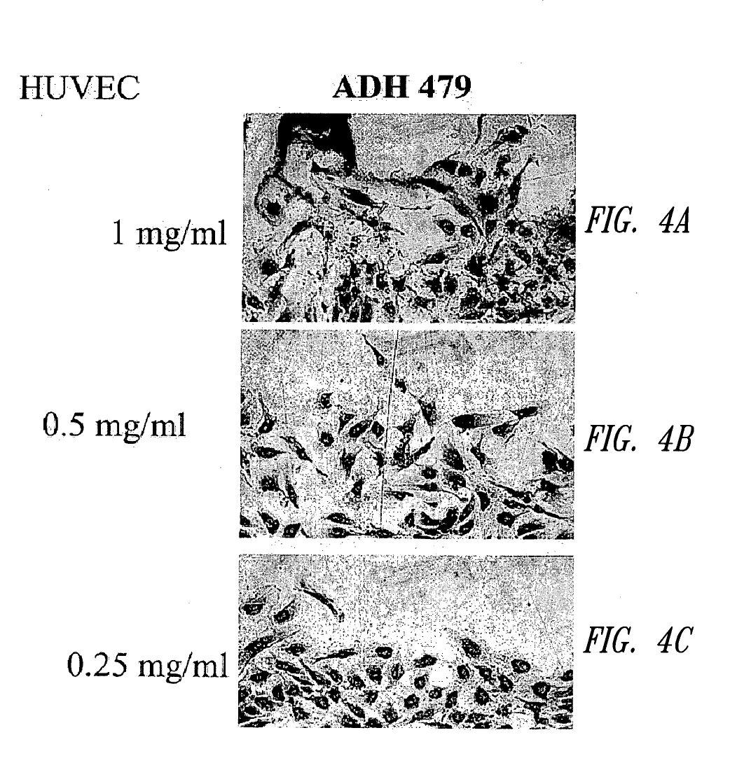

In control experiments (no peptide), the endothelial cell monolayer retained an adherent border along the edge of the wound. In the presence of the VE-cadherin-modulating peptide ADH479 (Ac-FRVDAETGDVFAIER-NH₂; SEQ ID NO: 18), the endothelial cells were observed to change morphology, reduce cell-cell contacts and migrate into the wound area (FIGS. 4A-C).

### Example 5

### Inhibition of Endothelial Tube Formation Upon Treatment with VE-Cadherin Peptide Modulating Agents

Endothelial cells were harvested from human umbilical vein and cultured as previously described (Lampugnani et al., 1995 JCB 129 203-217). Briefly, human endothelial cells were cultured on 1% gelatin-coated flask in medium 199 with 20% newborn calf serum (NCS) supplemented with 50 ug/mL endothelial growth supplement (ECGS) and 100 ug/mL heparin. Collagen tube formation assays were performed as previously described (Corada et al 2002 Blood 100 905-911; Corada et al 2001 Blood 97 1679-1684). Briefly, type I collagen (Collaborative Biomedical Product, Bedford, Mass.) from rat tail was diluted to a concentration of 1 mg/mL, and pH was neutralized by adding 1/10 volume of 10× minimal essential medium (MEM) (Life Technologies). Aliquots of 250 μL were added to each well of a 24-well culture plates and incubated at 37° C. until gelation occurred. Human umbilical vein endothelial cells were seeded on the collagen gel at 10,000 cells/mL in complete medium in the presence of peptides or controls for 24 hr. Medium was then removed and a second collagen gel coat was made on top of the cells (in a sandwich fashion) by adding soluble collagen for 30 min at 37° C. After this period, complete medium was added containing peptides or controls. Cells were left for 24 hr to form tubes. Tube formation was assessed by phase contrast microscopy.

In the presence of control (no peptide), endothelial cells formed tube-like structures within the collagen gel. These structures were observed by phase contrast microscopy and appear as connecting tube networks, with few single cells dispersed between the tubes. Incubation of the cells in the presence of either VE-cadherin-modulating peptides (1 mg/mL media) ADHI91 (Ac-CDAEC-NH₂; SEQ ID NO: A, ADH687 (Ac-H-CFRVDAC-OH; SEQ ID NO: _______), or ADH682 (Ac-CFRVDAETC-NH₂; SEQ ID NO: ______) reduced the incidence of tube-like structures. Large numbers of the endothelial cells remained dispersed in the collagen as single cells and did not form tube structures. This data indicates that the VE-cadherin-modulating peptides (Ac-CDAEC-NH₂; SEQ ID NO: ______), Ac-H-CFRVDAC-OH; SEQ ID NO: _______ and Ac-CFRVDAETC-NH₂; SEQ ID NO: ______) significantly inhibited the formation of endothelial tubes.

### Example 6

### Disruption of Human Umbilical Vein Endothelial Cell Adhersion Upon Treatment with VE-Cadherin Peptide Modulating Agents

Human umbilical vein endothelial cells (HUVEC) were obtained from Cambrex Bio Science Walkersville Inc. (Walkersville, Md.). HUVEC cells were cultured in endothelial growth media (EGM-2) supplemented with 2% FBS, hEGF, hydrocortizone, Gentamicin, Amphotericin-B, VEGF, hFGF-B, R³-IGF-1, ascorbic acid and heparin. The cells were kept in a humidified atmosphere (5% CO₂) at 37° C. All culture reagents were purchased from Cambrex Bio Science Walkersville Inc. (Walkersville, Md.).

Cells were exposed to cyclic peptides ADH142 (Ac-CDAEC-OH; SEQ ID NO: ______) or ADH191 (Ac-CDAEC-NH₂; SEQ ID NO: ______) at 1 mg/mL for 24 hr, and then fixed with 4% paraformaldehyde, followed by 3 washes with phosphate buffered saline (PBS) and staining with hematoxylin. Cells were viewed under light microscopy at 400×. The cyclic peptides ADH142 and ADH191 caused a perturbation of cell-cell contacts in the monolayer. The cells retracted from one another, and became spindle shaped with long processes. Large holes became apparent in the monolayer indicating a disruption of cell-cell adhesion.

### Example 7

### Disruption of Human Adult Microvasculature Endothelial Cells Upon Treatment with VE-Cadherin Peptide Modulating Agents

Dermal human adult microvascular endothelial cells (HMVEC-d) cells were obtained from Cambrex Bio Science Walkersville Inc. (Walkersville, Md.). HMVEC were cultured in endothelial cell media (EGM-2MV) supplemented with 5% FBS, hEGF, hydrocortizone, Gentamicin, Amphtotericin-B, VEGF, hFGF-B, R³-IGF-1 and ascorbic acid. The cells were kept in a humidified atmosphere (5% CO₂) at 37° C. All culture reagents were purchased from Cambrex Bio Science Walkersville Inc. (Walkersville, Md.).

Cells were exposed to cyclic peptides ADH142 (Ac-CDAEC-OH; SEQ ID NO: ______) or ADH191 (Ac-CDAEC-NH2; SEQ ID NO: ______) at 1 mg/mL for 24 hr, and then fixed with 4% paraformaldehyde, followed by 3 washes with phosphate buffered saline (PBS) and staining with hematoxylin. Cells were viewed under light microscopy at 400×. The cyclic peptides ADH 142 and ADH 191 caused a perturbation of cell-cell contacts in the monolayer. The cells retracted from one another and large holes became apparent in the monolayer indicating a disruption of cell-cell adhesion.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A modulating agent that:
(a) comprises the VE-cadherin cell adhesion recognition sequence DAE; and
(b) contains 3-16 amino acid residues linked by peptide bonds.

2. A modulating agent that:
(a) comprises at least five consecutive amino acid residues of a VE-cadherin cell adhesion recognition sequence having the formula:

| | |
|---|---|
| Aaa-Phe-Baa-Ile/Leu/Val-Asp-Ala-Glu- | (SEQ ID NO: 3) |
| Ser/Thr/Asn-Gly | |

wherein Aaa and Baa are independently selected amino acid residues; Ile/Leu/Val is an amino acid that is selected from the group consisting of isoleucine, leucine and valine, and Ser/Thr/Asn is an amino acid that is selected from the group consisting of serine, threonine and asparagine; and
(b) contains no more than 50 consecutive amino acid residues present within a naturally occurring VE-cadherin.

3. A modulating agent that:
(a) comprises at least seven consecutive amino acid residues of a VE-cadherin cell adhesion recognition sequence having the formula:

| | |
|---|---|
| Aaa-Phe-Baa-Ile/Leu/Val-Asp-Ala-Glu- | (SEQ ID NO: 3) |
| Ser/Thr/Asn-Gly | |

wherein Aaa and Baa are independently selected amino acid residues; Ile/LeuIVal is an amino acid that is selected from the group consisting of isoleucine, leucine and valine, and Ser/Thr/Asn is an amino acid that is selected from the group consisting of serine, threonine and asparagine; and
(b) contains no more than 50 consecutive amino acid residues present within a naturally occurring VE-cadherin.

4. A modulating agent that:
(a) comprises at least nine consecutive amino acid residues of an VE-cadherin cell adhesion recognition sequence having the formula:

| | |
|---|---|
| Aaa-Phe-Baa-Ile/Leu/Val-Asp-Ala-Glu- | (SEQ ID NO: 3) |
| Ser/Thr/Asn-Gly | |

wherein Aaa and Baa are independently selected amino acid residues; Ile/Leu/Val is an amino acid that is selected from the group consisting of isoleucine, leucine and valine, and Ser/Thr/Asn is an amino acid that is selected from the group consisting of serine, threonine and asparagine; and
(b) contains no more than 50 consecutive amino acid residues present within a naturally occurring VE-cadherin.

5. A modulating agent according to any one of claims 1-4, wherein the agent is a peptide ranging in size from 3 to 50 amino acid residues.

6. A modulating agent according to any one of claims 1-4, wherein the agent is a peptide ranging in size from 4 to 16 amino acid residues.

7. A modulating agent according to any one of claims 1-4, wherein the cell adhesion recognition sequence is present within a cyclic peptide.

8. A modulating agent according to claim 7, wherein the cyclic peptide has the formula: 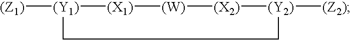
wherein W is the amino acid sequence DAE;
wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12;
wherein Y₁ and Y₂ are independently selected from the group consisting of amino acid residues, and wherein a covalent bond is formed between residues Y₁ and Y₂; and
wherein Z₁ and Z₂ are optional, and if present, are independently selected from the group consisting of amino acid residues and combinations thereof in which the residues are linked by peptide bonds.

9. A polynucleotide encoding a modulating agent according to any one of claims 1-4.

10. An expression vector comprising a polynucleotide according to claim 9.

11. A host cell transformed or transfected with an expression vector according to claim 10.

12. A modulating agent comprising an antibody or antigen-binding fragment thereof that specifically binds to a VE-cadherin cell adhesion recognition sequence set forth in any one of claims 1-4, wherein the agent is capable of modulating a VE-cadherin-mediated function.

13. A modulating agent comprising a mimetic of a VE-cadherin cell adhesion recognition sequence set forth in any one of claims 1-4, wherein the agent is capable of modulating a VE-cadherin-mediated function.

14. A modulating agent according to any one of claims 1-4, wherein the agent comprises the VE-cadherin cell adhesion recognition sequence FRVDAETG (SEQ ID NO: 14)

15. A modulating agent according to claim 14, wherein the agent comprises a linear peptide having the sequence Ac-FRVDAETGDVFAIER-NH2 (SEQ ID NO: 18) or N-Ac-VFRVDAETGD-NH₂ (SEQ ID NO: 19).

16. A modulating agent according to claim 14, wherein a VE-cadherin cell adhesion recognition sequence is present within a cyclic peptide.

17. A modulating agent according to claim 16, wherein the cyclic peptide comprises a sequence selected from the group consisting of CDAEC (SEQ ID NO: 30), CVDAEC (SEQ ID NO: 31), CDAETC (SEQ ID NO: 32), CRVDAEC (SEQ ID NO: 33), CVDAETC (SEQ ID NO: 34), CRVDAETC (SEQ ID NO: 35), CDAETGC (SEQ ID NO: 36), CCDAETGC (SEQ ID NO: 37), CRVDAETGC (SEQ ID NO: 38), CFRVDAEC (SEQ ID NO: 39), CFRVDAETC (SEQ ID NO: 40), CFRVDAETGC (SEQ ID NO: 41), CVFRVDAEC (SEQ ID NO: 42), CVFRVDAETC (SEQ ID NO: 43), CVFRVDAETGC (SEQ ID NO: 44), DDAEK (SEQ ID NO: 45), DVDAEK (SEQ ID NO: 46), DRVDAEK (SEQ ID NO: 47), DFRVDAEK (SEQ ID NO: 48), DVFRVDAEK (SEQ ID NO: 49), EDAEK (SEQ ID NO: 50), EVDAEK (SEQ ID NO: 51), ERVDAEK (SEQ ID NO: 52), EFRVDAEK (SEQ ID NO: 53), EVFRVDAEK (SEQ ID NO: 54), KDAED (SEQ ID NO: 55), KVDAED (SEQ ID NO: 56), KDAETD (SEQ ID NO: 57), KRVDAED(SEQ ID NO: 58), KVDAETD (SEQ ID NO: 59), KRVDAETD (SEQ ID NO: 60), KDAETGD (SEQ ID NO: 61), KVDAETGD (SEQ ID NO: 62), KRVDAETGD (SEQ ID NO: 63), KFRVDAED (SEQ ID NO: 64), KFRVDAETD (SEQ ID NO: 65), KFRVDAETGD (SEQ ID NO: 66), KVFRVDAED (SEQ ID NO: 67), KVFRVDAETD (SEQ ID NO: 68), KVFRVDAETGD (SEQ ID NO: 69), VDAEK (SEQ ID NO: 70), IDAES (SEQ ID NO: 71), VDAES (SEQ ID NO: 72), DAETG (SEQ ID NO: 73), VDAETG (SEQ ID NO: 74), KDAEE (SEQ ID NO: 75), KVDAE (SEQ ID NO: 76), KDAETE (SEQ ID NO: 77), KRVDAE (SEQ ID NO: 78), KVDAETE (SEQ ID NO: 79), KRVDAETE (SEQ ID NO: 80), KDAETGE (SEQ ID NO: 81), KVDAETGE (SEQ ID NO: 82), KRVDAETGE (SEQ ID NO: 83), KFRVDAE (SEQ ID NO: 84), KFRVDAETE (SEQ ID NO: 85), KFRVDAETGE (SEQ ID NO: 86), KVFRVDAE (SEQ ID NO: 87), KVFRVDAETE (SEQ ID NO: 88), KVFRVDAETGE (SEQ ID NO: 89), VDAET (SEQ ID NO: 90), VDAETG (SEQ ID NO: 91), DAETG (SEQ ID NO: 92), RVDAE (SEQ ID NO: 93), RVDAET (SEQ ID NO: 94), RVDAETG (SEQ ID NO: 95), FRVDAE (SEQ ID NO: 96), FRVDAET (SEQ ID NO: 97), FRVDAETG (SEQ ID NO: 98), VFRVDAE (SEQ ID NO: 99), VFRVDAET (SEQ ID NO: 100), VFRVDAETG (SEQ ID NO: 101), FRV, RVD, VDA, FRVDA (SEQ ID NO: ______), RVDA (SEQ ID NO: ______), CVDAC (SEQ ID NO: ______), CFRVC (SEQ ID NO: ______), CRVDC (SEQ ID NO: ______), CVDAC (SEQ ID NO: ______), CFRVDAC (SEQ ID NO: ______), CRVDAC (SEQ ID NO: ______) and CVDAC (SEQ ID NO: ______).

18. A polynucleotide encoding a modulating agent according to claim 14.

19. A modulating agent comprising an antibody or antigen-binding fragment thereof that specifically binds to a VE-cadherin cell adhesion recognition sequence set forth in any one of claims 1-4, wherein the agent modulates a VE-cadherin-mediated function.

20. A modulating agent according to any one of claims 1-4 linked to a drug.

21. A modulating agent according to any one of claims 1-4 linked to a detectable marker.

22. A modulating agent according to any one of claims 1-4 linked to a targeting agent.

23. A modulating agent according to any one of claims 1-4 linked to a support material.

24. A modulating agent according to claim 23, wherein the support material is a polymeric matrix.

25. A modulating agent according to claim 23, wherein the support material is selected from the group consisting of plastic dishes, plastic tubes, sutures, membranes, ultra thin films, bioreactors and microparticles.

26. A modulating agent according to any one of claims 1-4, further comprising one or more of:
(a) a cell adhesion recognition sequence that is specifically recognized by an adhesion molecule other than a VE-cadherin; and/or
(b) an antibody or antigen-binding fragment thereof that specifically binds to a cell adhesion recognition sequence that is specifically recognized by an adhesion molecule other than a VE-cadherin.

27. A modulating agent according to claim 26, wherein the adhesion molecule is selected from the group consisting of cadherins, integrins, occludin, claudins, desmogleins, desmocollins, protocadherins, cadherin-related neuronal receptors, claudins, N-CAM, JAM, CEA, L1 fibronectin, laminin, and other extracellular matrix proteins.

28. A pharmaceutical composition comprising a modulating agent according to any one of claims 1-4 in combination with a pharmaceutically acceptable carrier.

29. A composition according to claim 28, further comprising a drug.

30. A composition according to claim 28, wherein the modulating agent is present within a sustained-release formulation.

31. A pharmaceutical composition according to claim 28, further comprising a modulator of cell adhesion that comprises one or more of:
(a) a cell adhesion recognition sequence that is specifically recognized by an adhesion molecule other than a VE-cadherin; and/or
(b) an antibody or antigen-binding fragment thereof that specifically binds to a cell adhesion recognition sequence that is specifically recognized by an adhesion molecule other than a VE-cadherin.

32. A pharmaceutical composition according to claim 31, wherein the adhesion molecule is selected from the group consisting of cadherins, integrins, occludin, claudins, desmogleins, desmocollins, protocadherins, cadherin-related neuronal receptors, N-CAM, JAM, CEA, L1, fibronectin, laminin and other extracellular matrix proteins.

33. A method for modulating cell adhesion comprising contacting a VE-cadherin-expressing cell with a modulating agent according to claim 1, and thereby enhancing cell adhesion.

34. A method for modulating angiogenesis comprising contacting a VE-cadherin-expressing cell with a modulating agent according to claim 1, and thereby modulating angiogenesis.

35. A method for modulating endothelial cell adhesion, comprising contacting a VE-cadherin expressing cell with a modulating agent according to claim 1, and thereby modulating endothelial cell adhesion.

36. A method for stimulating blood vessel regression, comprising contacting a VE-cadherin-expressing blood vessel with a modulating agent according to claim 1, and thereby stimulating blood vessel regression.

37. A method for disrupting neovasculature in a mammal, comprising contacting a VE-cadherin expressing cell with a modulating agent according to claim 1, and thereby disrupting neovasculature.

38. A method for increasing vasopermeability in a mammal, comprising contacting a VE-cadherin-expressing endothelial cell with a modulating agent according to claim 1, and thereby increasing vasopermeability.

39. A method for facilitating blood sampling in a mammal, comprising contacting a VE-cadherin expressing cell with a modulating agent according to claim 1, and thereby facilitating blood sampling.

40. A method for treating cancer in a mammal, comprising administering to a mammal a modulating agent according to claim 1, and thereby treating cancer.

41. A method for reducing the size of a tumor in a mammal, comprising administering to a mammal a modulating agent according to claim 1, and thereby reducing the size of the tumor.

42. A method for treating metastasis in a mammal, comprising administering to a mammal a modulating agent according to claim 1, and thereby treating metastasis.

43. A method for enhancing the delivery of a drug to a tumor in a mammal, comprising administering to a mammal a modulating agent according to claim 1, and thereby enhancing delivery of a drug to the tumor.

44. A method for modulating a tumor permeability barrier to drugs, comprising contacting a VE-cadherin-expressing cell with a modulating agent according to claim 1, and thereby modulating a tumor permeability barrier.

45. A method for enhancing drug delivery to the central nervous system of a mammal comprising administering to a mammal a modulating agent according to claim 1, and thereby enhancing drug delivery to the central nervous system.

46. A method for modulating apoptosis in a cell, comprising contacting a VE-cadherin-expressing cell with a modulating agent according to claim 1, and thereby modulating apoptosis.

47. A method for facilitating wound healing, comprising contacting a VE-cadherin-expressing cell with a modulating agent according to claim 1, and thereby facilitating wound healing.

48. A method for enhancing adhesion of foreign tissue implanted within a mammal, comprising contacting a site of implantation of foreign tissue in a mammal with a modulating agent according to claim 1, and thereby enhancing adhesion of the foreign tissue.

49. A method for modulating the immune system of a mammal, comprising administering to a mammal a modulating agent according to claim 1, wherein the modulating agent inhibits VE-cadherin-mediated cell adhesion, and thereby modulating the immune system of a mammal.

50. A method for preventing pregnancy in a mammal, comprising administering to a mammal a modulating agent according to claim 1, wherein the modulating agent inhibits VE-cadherin-mediated cell adhesion, and thereby preventing pregnancy in a mammal.

51. A method for preventing or treating obesity comprising administering to a mammal a modulating agent according to claim 1, and thereby preventing or treating obesity.

52. A modulating agent that:
(a) comprises the VE-cadherin cell adhesion recognition sequence DAN, DKN or DEN; and
(b) contains 3-16 amino acid residues linked by peptide bonds.

53. A modulating agent that:
(a) comprises the VE-cadherin cell adhesion recognition sequence FRV, RVD or VDA; and
(b) contains 3-16 amino acid residues linked by peptide bonds.

54. A modulating agent comprising a cyclic peptide, wherein the cyclic peptide has the formula: 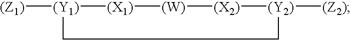
wherein W is the amino acid sequence DAN, DKN or DEN;
wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12;
wherein Y₁ and Y₂ are independently selected from the group consisting of amino acid residues, and wherein a covalent bond is formed between residues Y₁ and Y₂; and
wherein Z₁ and Z₂ are optional, and if present, are independently selected from the group consisting of amino acid residues and combinations thereof in which the residues are linked by peptide bonds.

55. A modulating agent comprising a cyclic peptide, wherein the cyclic peptide has the formula: 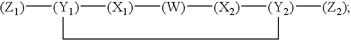
wherein W is the amino acid sequence FRV, RVD or VDA;
wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12;
wherein Y₁ and Y₂ are independently selected from the group consisting of amino acid residues, and wherein a covalent bond is formed between residues Y₁ and Y₂; and
wherein Z₁ and Z₂ are optional, and if present, are independently selected from the group consisting of amino acid residues and combinations thereof in which the residues are linked by peptide bonds.
